SEQUENCE LISTING (96 sequences)

SEQ ID NO: 1 - protein, 232 aa, Homo sapiens
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 115 aa, Artificial sequence
  Y035 VH
EVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSA

SEQ ID NO: 3 - protein, 113 aa, Artificial sequence
  Y035 VL
DIVMTQTPLSLPVITGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKR

SEQ ID NO: 4 - protein, 475 aa, Artificial sequence
  Y035 scFv-hFc
DIVMTQTPLSLPVITGEPASISCRSSQSLVHSNGNTYLQWYLQKPGQSPQLLIYKVSNRFSGVPDRFSGSGSGTDFTLKISRVEAEDVGVYYCSQSIYVPYTFGQGTKLEIKRGGGGSGGGGSGGGGSEVQLVQSGAEVKKPGASVKVSCKASGYTFSDYEMHWVRQAPGQGLEWMGAIHPGSGDTAYNQRFKGRVTITADKSTSTAYMELSSLRSEDTAVYYCARFYSYAYWGQGTLVTVSAEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 121 aa, Artificial squence
  T2-23 VH
QVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSS

SEQ ID NO: 6 - protein, 111 aa, Artificial sequence
  T2-23 VL
QSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLG

SEQ ID NO: 7 - protein, 479 aa, Artificial sequence
  T2-23 scFv-hFc
QSALTQPPSASGSPGQSVTISCTGTSSDVGGYNYVSWYQQYPGKAPKLLIYGNSNRPSGVPDRFSGSKSGTSASLAITGLQAEDGADYYCQSYDSSLRVVFGGGTKVTVLGGGGGSGGGGSGGGGSQVQLQESGGGLVQPGRSLRLSCAASGFTFSSYAMHWVRQAPGKGLEWVSAISSSGRSTYYADSVEGRFTISRDNSKNTLYLQMNSLRAEDTAVYYCAKDRRGSHADALNVWGQGTLVTVSSEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 40 aa, Artificial sequence
  human GPC3 Ala524-Lys563
AELAYDLDVDDAPGNSQQATPKDNEISTFHNLGNVHSPLK

SEQ ID NO: 9 - protein, 50 aa, Artificial sequence
  human GPC3Asp511-Ser560
DGMIKVKNQLRFLAELAYDLDVDDAPGNSQQATPKDNEISTFHNLGNVHS

SEQ ID NO: 10 - protein, 580 aa, Homo sapiens
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSMPKGRVLDKNLDEEGFESGDCGDDEDECIGGSGDGMIKVKNQLRFLAELAYDLDVDDAPGNSQQATPKDNEISTFHNLGNVHSPLKLLTSMAISVVCFFFLVH

SEQ ID NO: 11 - protein, 580 aa, Macaca fascicularis
MAGTVRTACLVVAMLLSLDFPGQAQPPPPPPDATCHQVRSFFQRLQPGLKWVPETPVPGSDLQVCLPKGPTCCSRKMEEKYQLTARLNMEQLLQSASMELKFLIIQNAAVFQEAFEIVVRHAKNYTNAMFKNNYPSLTPQAFEFVGEFFTDVSLYILGSDINVDDMVNELFDSLFPVIYTQLMNPGLPDSALDINECLRGARRDLKVFGNFPKLIMTQVSKSLQVTRIFLQALNLGIEVINTTDHLKFSKDCGRMLTRMWYCSYCQGLMMVKPCGGYCNVVMQGCMAGVVEIDKYWREYILSLEELVNGMYRIYDMENVLLGLFSTIHDSIQYVQKNAGKLTTTIGKLCAHSQQRQYRSAYYPEDLFIDKKVLKVAHVEHEETLSSRRRELIQKLKSFISFYSALPGYICSHSPVAENDTLCWNGQELVERYSQKAARNGMKNQFNLHELKMKGPEPVVSQIIDKLKHINQLLRTMSVPKGRVLDKNLDEEGFESGDCGDDEDECIGGSGDGMMKVKNQLRFLAELAYDLDVDDVPGNNQQATPKDNEISTFHNLGNVHSPLKLLTSMAISVVCFFFLVH

SEQ ID NO: 12 - DNA, 18 bp, Artificial sequence
  foward primer
cttggtggtcctggctgc

SEQ ID NO: 13 - DNA, 23 bp, Artificial sequence
  reverse primer
ggtacgtgctgttgaactgttcc

SEQ ID NO: 14 - DNA, 47 bp, Artificial sequence
  forward sequnce
catgccatgactgtggcccaggcggcccagktgcagctcgtggagtc

SEQ ID NO: 15 - DNA, 51 bp, Artificial sequnce
  reverse primer
catgccatgactcgcggccggcctggccatgggggtcttcgctgtggtgcg

SEQ ID NO: 16 - DNA, 52 bp, Artificial sequence
  reverse sequence
catgccatgactcgcggccggcctggccgtcttgtggttttggtgtcttggg

SEQ ID NO: 17 - protein, 123 aa, Vicugna pacos
QVQLVESGGGLVQAGGSLKLSCAASGRSFSPYAMGWFRQAPGKDREFVAAISNSGGSTYYADAVKGRFSISRDNAKNTVYLQMNNLEPEDTAVYYCTGPVKRYSTDFQGGDYWGQGTQVTVSS

SEQ ID NO: 18 - protein, 122 aa, Vicugna pacos
QVQLVESGGGLVQPGGSLRLSCAASGSISTYTMAWYRQAPGEQRESVAAISSGGRTDYIDSVKGRFTISRDNAKNMVYLQMNSLKPEDTAVYYCNSADGLKIGTYYFKGLGWGQGTQVTVSA

SEQ ID NO: 19 - protein, 123 aa, Vicugna pacos
QVQLVESGGGLVRPGGSLRVSCAASGVDISTYTMAWYRQAPGEQRESVAAISTTGRSIYIDAVQGRFTMSRDNAKNTVYLQMNNLKPEDTAVYYCNSADDLKIGTQYFKGLGWGQGTQVTVSS

SEQ ID NO: 20 - protein, 124 aa, Vicugna pacos
QVQLVESGGGLVQAGGSLSLSCAASGRRFSTNVMGWFRQAPGKEREFLAAINWVIGNTNYAESVKGRFTISRDNAKETVYLQMDNLKVEDTAVYYCAGRSSYYTSSRREDYDYWGQGTQVTVSS

SEQ ID NO: 21 - protein, 123 aa, Vicugna pacos
QVQLVESGGGLVQAGASLRLSCLGSGRSFGLRAMGWFRQAPGKELEFVAAIGKAGDTTYYTDSVKGRFTISRDNVKNAVYLQMNSLKPEDTAVYVCATAARWEPPTITPGSYRGPGTQVTVSS

SEQ ID NO: 22 - protein, 121 aa, Vicugna pacos
QVQLVESGGGLVQAGGSLRLSCAASGSILSITAMNWHRQAPGKERELVASITNGGRTNYADSVKGRFTISRDRAKGTLYLQMNNLKPEDTAVYYCYARRWTGGDRPEYESWGQGTQVTVSS

SEQ ID NO: 23 - protein, 123 aa, Vicugna pacos
QLQLVESGGGLVQPGGSLRLSCAASGFTLDVYAIGWFRQAPGKEREGVSYITSGDGIFYADSVKGRFTISRDNAKNTVYLQMNSLKPEDTAVYFCATLKRTTAGGWPIPGRIGGQGTQVTVSS

SEQ ID NO: 24 - protein, 8 aa, Artificial sequence
  CDR
GRSFSPYA

SEQ ID NO: 25 - protein, 8 aa, Artificial sequence
  CDR
ISNSGGST

SEQ ID NO: 26 - protein, 16 aa, Artificial sequence
  CDR
TGPVKRYSTDFQGGDY

SEQ ID NO: 27 - protein, 5 aa, Artificial sequence
  CDR
PYAMG

SEQ ID NO: 28 - protein, 17 aa, Artificial sequence
  CDR
AISNSGGSTYYADAVKG

SEQ ID NO: 29 - protein, 14 aa, Artificial sequence
  CDR
PVKRYSTDFQGGDY

SEQ ID NO: 30 - protein, 7 aa, Artificial sequence
  CDR
GRSFSPY

SEQ ID NO: 31 - protein, 6 aa, Artificial sequence
  CDR
SNSGGS

SEQ ID NO: 32 - protein, 14 aa, Artificial sequence
  CDR
PVKRYSTDFQGGDY

SEQ ID NO: 33 - protein, 7 aa, Artificial sequence
  CDR
GSISTYT

SEQ ID NO: 34 - protein, 7 aa, Artificial sequence
  CDR
ISSGGRT

SEQ ID NO: 35 - protein, 17 aa, Artificial sequence
  CDR
NSADGLKIGTYYFKGLG

SEQ ID NO: 36 - protein, 4 aa, Artificial sequence
  CDR
YTMA

SEQ ID NO: 37 - protein, 16 aa, Artificial sequence
  CDR
AISSGGRTDYIDSVKG

SEQ ID NO: 38 - protein, 15 aa, Artificial sequence
  CDR
ADGLKIGTYYFKGLG

SEQ ID NO: 39 - protein, 6 aa, Artificial sequence
  GSISTY
GSISTY

SEQ ID NO: 40 - protein, 5 aa, Artificial sequence
  CDR
SSGGR

SEQ ID NO: 41 - protein, 15 aa, Artificial sequence
  CDR
ADGLKIGTYYFKGLG

SEQ ID NO: 42 - protein, 8 aa, Artificial sequence
  CDR
GVDISTYT

SEQ ID NO: 43 - protein, 7 aa, Artificial sequence
  CDR
ISTTGRS

SEQ ID NO: 44 - protein, 17 aa, Artificial sequence
  CDR
NSADDLKIGTQYFKGLG

SEQ ID NO: 45 - protein, 5 aa, Artificial sequence
  CDR
TYTMA

SEQ ID NO: 46 - protein, 16 aa, Artificial sequence
  CDR
AISTTGRSIYIDAVQG

SEQ ID NO: 47 - protein, 15 aa, Artificial sequence
  CDR
ADDLKIGTQYFKGLG

SEQ ID NO: 48 - protein, 7 aa, Artificial sequence
  CDR
GVDISTY

SEQ ID NO: 49 - protein, 5 aa, Artificial sequence
  CDR
STTGR

SEQ ID NO: 50 - protein, 15 aa, Artificial sequence
  CDR
ADDLKIGTQYFKGLG

SEQ ID NO: 51 - protein, 8 aa, Artificial sequence
  CDR
GRRFSTNV

SEQ ID NO: 52 - protein, 8 aa, Artificial sequence
  CDR
INWVIGNT

SEQ ID NO: 53 - protein, 17 aa, Artificial sequence
  CDR
AGRSSYYTSSRREDYDY

SEQ ID NO: 54 - protein, 5 aa, Artificial sequence
  CDR
TNVMG

SEQ ID NO: 55 - protein, 17 aa, Artificial sequence
  CDR
AINWVIGNTNYAESVKG

SEQ ID NO: 56 - protein, 15 aa, Artificial sequence
  CDR
RSSYYTSSRREDYDY

SEQ ID NO: 57 - protein, 7 aa, Artificial sequence
  CDR
GRRFSTN

SEQ ID NO: 58 - protein, 6 aa, Artificial sequence
  CDR
NWVIGN

SEQ ID NO: 59 - protein, 15 aa, Artificial sequence
  CDR
RSSYYTSSRREDYDY

SEQ ID NO: 60 - protein, 8 aa, Artificial sequence
  CDR
GRSFGLRA

SEQ ID NO: 61 - protein, 8 aa, Artificial sequence
  CDR
IGKAGDTT

SEQ ID NO: 62 - protein, 12 aa, Artificial sequence
  CDR
ATAARWEPPTIT

SEQ ID NO: 63 - protein, 5 aa, Artificial sequence
  CDR
LRAMG

SEQ ID NO: 64 - protein, 17 aa, Artificial sequence
  CDR
AIGKAGDTTYYTDSVKG

SEQ ID NO: 65 - protein, 14 aa, Artificial sequence
  CDR
AARWEPPTITPGSY

SEQ ID NO: 66 - protein, 7 aa, Artificial sequence
  CDR
GRSFGLR

SEQ ID NO: 67 - protein, 6 aa, Artificial sequence
  CDR
GKAGDT

SEQ ID NO: 68 - protein, 14 aa, Artificial sequence
  CDR
AARWEPPTITPGSY

SEQ ID NO: 69 - protein, 8 aa, Artificial sequence
  CDR
GSILSITA

SEQ ID NO: 70 - protein, 7 aa, Artificial sequence
  CDR
ITNGGRT

SEQ ID NO: 71 - protein, 15 aa, Artificial sequence
  CDR
YARRWTGGDRPEYES

SEQ ID NO: 72 - protein, 5 aa, Artificial sequence
  CDR
ITAMN

SEQ ID NO: 73 - protein, 16 aa, Artificial sequence
  CDR
SITNGGRTNYADSVKG

SEQ ID NO: 74 - protein, 13 aa, Artificial sequence
  CDR
RRWTGGDRPEYES

SEQ ID NO: 75 - protein, 7 aa, Artificial sequence
  CDR
GSILSIT

SEQ ID NO: 76 - protein, 5 aa, Artificial sequence
  CDR
TNGGR

SEQ ID NO: 77 - protein, 13 aa, Artificial sequence
  CDR
RRWTGGDRPEYES

SEQ ID NO: 78 - protein, 8 aa, Artificial sequence
  CDR
GFTLDVYA

SEQ ID NO: 79 - protein, 7 aa, Artificial sequence
  CDR
ITSGDGI

SEQ ID NO: 80 - protein, 12 aa, Artificial sequence
  CDR
ATLKRTTAGGWP

SEQ ID NO: 81 - protein, 5 aa, Artificial sequence
  CDR
VYAIG

SEQ ID NO: 82 - protein, 16 aa, Artificial sequence
  CDR
YITSGDGIFYADSVKG

SEQ ID NO: 83 - protein, 15 aa, Artificial sequence
  CDR
LKRTTAGGWPIPGRI

SEQ ID NO: 84 - protein, 7 aa, Artificial sequence
  CDR
GFTLDVY

SEQ ID NO: 85 - protein, 5 aa, Artificial sequence
  CDR
TSGDG

SEQ ID NO: 86 - protein, 15 aa, Artificial sequence
  CDR
LKRTTAGGWPIPGRI

SEQ ID NO: 87 - protein, 124 aa, Vicugna pacos
QVQLVESGGGLVQAGGSLRLSCAASGFTLENYAIGWFRQAPGKEREGVSYITGGTGTTVYADSVKGRFTISRDNTKNTVYLQMNSLKPEDTAVYFCATLKRTTAGGWPKPGRIGGQGTQVTVSS

SEQ ID NO: 88 - protein, 8 aa, Artificial sequence
  CDR
GFTLENYA

SEQ ID NO: 89 - protein, 8 aa, Artificial sequence
  CDR
ITGGTGTT

SEQ ID NO: 90 - protein, 12 aa, Artificial sequence
  CDR
ATLKRTTAGGWP

SEQ ID NO: 91 - protein, 5 aa, Artificial sequence
  CDR
NYAIG

SEQ ID NO: 92 - protein, 17 aa, Artificial sequence
  CDR
YITGGTGTTVYADSVKG

SEQ ID NO: 93 - protein, 15 aa, Artificial sequence
  CDR
LKRTTAGGWPKPGRI

SEQ ID NO: 94 - protein, 7 aa, Artificial sequence
  CDR
GFTLENY

SEQ ID NO: 95 - protein, 6 aa, Artificial sequence
  CDR
TGGTGT

SEQ ID NO: 96 - protein, 15 aa, Artificial sequence
  CDR
LKRTTAGGWPKPGRI

(19) United States
(12) Patent Application Publication
(10) Pub. No.: US 2024/0052055 A1
(43) Pub. Date: Feb. 15, 2024

(54) GPC3 ANTIBODY AND APPLICATION THEREOF

(71) Applicant: Simcere Zaiming Pharmaceutical Co., Ltd., Haikou, Hainan (CN)

(72) Inventors: Shumei You, Shanghai (CN); Cuiqing Yang, Shanghai (CN); Zhuoxiao Cao, Shanghai (CN); Renhong Tang, Shanghai (CN); Jinsheng Ren, Nanjing, Jiangsu (CN)

(21) Appl. No.: 18/266,393

(22) PCT Filed: Dec. 9, 2021
(86) PCT No.: PCT/CN2021/136637

§ 371 Date: Jun. 9, 2023

(30) Foreign Application Priority Data
Dec. 10, 2020 (CN) 202011437242.9
Nov. 16, 2021 (CN) 202111353420.4

(51) Int. Cl. C07K 16/30; A61K 39/00; G01N 33/574

(52) U.S. Cl. CPC C07K 16/303; A61K 39/464474; G01N 33/57492; A61K 39/4631; C07K 2317/92; C07K 2317/33; C07K 2317/31; C07K 2317/22; C07K 2317/569; C07K 2317/24; A61K 2239/13

(57) ABSTRACT

Provided are a GPC3 antibody and an application thereof, and specifically provided are an antibody or an antigen-binding fragment that specifically binds to GPC3, a multispecific antigen-binding molecule, a chimeric antigen receptor, an immune effector cell, isolated nucleic acid fragments, a vector, a host cell, a corresponding preparation method, a pharmaceutical composition, a treatment method, a pharmaceutical use, a GPC3 detection method and a detection kit. The present disclosure is of great significance in the preparation of drugs for treating cancer or tumor.

The present disclosure claims priority to the Chinese Patent Application No. 202011437242.9 entitled “GPC3 ANTIBODY AND APPLICATION THEREOF”, filed with the China National Intellectual Property Administration on Dec. 10, 2020, which is incorporated herein by reference in its entirety. The present disclosure further claims priority to Chinese Patent Application No. 202111353420.4 entitled “GPC3 ANTIBODY AND APPLICATION THEREOF”, filed with the China National Intellectual Property Administration on Nov. 16, 2021, which is incorporated herein by reference in its entirety.

### TECHNICAL FIELD

The present disclosure relates to the field of antibodies, and in particular, to a GPC3 antibody and application thereof.

### BACKGROUND

Glypican-3 (GPC3) is a heparan sulfate (HS) glycoprotein. As a member of the heparan sulfate proteoglycans, GPC3 is anchored to cell membrane by glycophosphatidylinositol (GPI). The GPC3 core protein consists of 580 amino acids and is about 70 kD in size. The protein can be cleaved by furin to produce a 40 kD N-terminal subunit and a 30 kD C-terminal subunit, and the two subunits are linked through disulfide bonds. Two H S side chains of GPC3 are attached at a portion near the C terminus (Takahiro Nishida, Hiroaki Kataoka. Glypican 3-Targeted Therapy in Hepatocellular Carcinoma, Cancers 2019; 11(9):1339).

GPC3 plays a crucial regulatory role in cellular proliferation in mesodermal tissues, and deletion of the GPC3 gene will lead to Simpson-Golabi-Behmel syndrome (SGBS). GPC3 is expressed throughout the fetal period, but is seldom expressed after birth in normal tissues despite the weak expression in placental, mammary, mesothelial, ovarian, lung and kidney tissues. Abnormal expression of GPC3 has been reported in various tumor tissues in adults, such as hepatocellular carcinoma (HCC), lung squamous cell carcinoma, gastric cancer, and ovarian cancer. Particularly, GPC3 is overexpressed in HCC cells. It promotes the growth and invasion of HCC cells by enhancing autocrine/paracrine canonical Wnt signaling (Capurro M I, Xiang Y-Y, Lobe C, Filmus J. Glypican-3 promotes the growth of hepatocellular carcinoma by stimulating canonical Wnt signaling. Cancer Res 2005; 65:6245-54.). Immunohistochemistry revealed overexpression of GPC3 in approximately 70% of tumor tissues from HCC patients (Capurro M, Wanless I R, Sherman M, et al. Glypican-3: a novel serum and histochemical marker for hepatocellular carcinoma. Gastroenterology 2003; 125:89-97). Therefore, GPC3 is believed to be a candidate target for tumor therapy.

In 1993, Belgian scientist Hamers-Casterman C found a natural heavy chain antibody devoid of light chains in camel blood. Compared with ordinary antibodies, the heavy chain antibody still retains the ability to bind antigens despited lack of light chains (Hamers-Casterman C, Atarhouch T, Muyldermans S, Robinson G, Hamers C, Songa E B, et al. Naturally occurring antibodies devoid of light chains. Nature. 363(6428):446-8 (1993)). By cloning the variable region of the heavy chain antibody from camels, a single domain antibody (sdAb) consisting of only one heavy chain variable region is obtained, also referred to as a nanobody or a VHH domain (variable heavy chain domain of a heavy chain antibody).

Compared with ordinary antibodies, the nanobody has the advantages of low molecular weight, good stability, high solubility, and the like. Therefore, the development of GPC3 antibodies by nanobody technology has broad prospects, and the derivation of GPC3-specific nanobodies is of great significance in the development of related therapeutics or assay reagents.

### SUMMARY

The present disclosure discloses an antibody or an antigen-binding fragment specifically binding to GPC3, a polypeptide, a chimeric antigen receptor, an immune effector cell, an isolated nucleic acid fragment, a vector, a host cell, a method for preparing the same, a pharmaceutical composition, a treatment method, pharmaceutical use, a method for detecting GPC3, and an assay kit.

In a first aspect, the present disclosure relates to an antibody or an antigen-binding fragment specifically binding to GPC3 comprising a CDR1, a CDR2, and a CDR3.

In some specific embodiments, the CDR1, the CDR2, and the CDR3 are an HCDR1, an HCDR2, and an HCDR3, respectively.

In some specific embodiments, the HCDR1, the HCDR2, and the HCDR3 are determined according to the IMGT numbering system, the Kabat numbering system, or the Chothia numbering system, e.g., selected from Table 1; for example, the HCDR1 is selected from SEQ ID NOs: 24, 27, 30, 33, 36, 39, 42, 45, 48, 51, 54, 57, 60, 63, 66, 69, 72, 75, 78, 81, 84, 88, 91, and 94, the HCDR2 is selected from SEQ ID NOs: 25, 28, 31, 34, 37, 40, 43, 46, 49, 52, 55, 58, 61, 64, 67, 70, 73, 76, 79, 82, 85, 89, 92, and 95, and the HCDR3 is selected from SEQ ID NOs: 26, 29, 32, 35, 38, 41, 44, 47, 50, 53, 56, 59, 62, 65, 68, 71, 74, 77, 80, 83, 86, 90, 93, and 96;
preferably, according to the IMGT numbering system, the Kabat numbering system, or the Chothia numbering system, the HCDR1, the HCDR2, and the HCDR3 are selected from sequences set forth in SEQ ID NOs: 24-26, 27-29, and 30-32, respectively;
preferably, according to the IMGT numbering system, the Kabat numbering system, or the Chothia numbering system, the HCDR1, the HCDR2, and the HCDR3 are selected from sequences set forth in SEQ ID NOs: 33-35, 36-38, and 39-41, respectively;
preferably, according to the IMGT numbering system, the Kabat numbering system, or the Chothia numbering system, the HCDR1, the HCDR2, and the HCDR3 are selected from sequences set forth in SEQ ID NOs: 42-44, 45-47, and 48-50, respectively;
preferably, according to the IMGT numbering system, the Kabat numbering system, or the Chothia numbering system, the HCDR1, the HCDR2, and the HCDR3 are selected from sequences set forth in SEQ ID NOs: 51-53, 54-56, and 57-59, respectively;
preferably, according to the IMGT numbering system, the Kabat numbering system, or the Chothia numbering system, the HCDR1, the HCDR2, and the HCDR3 are selected from sequences set forth in SEQ ID NOs: 60-62, 63-65, and 66-68, respectively;
preferably, according to the IMGT numbering system, the Kabat numbering system, or the Chothia numbering system, the HCDR1, the HCDR2, and the HCDR3 are selected from sequences set forth in SEQ ID NOs: 69-71, 72-74, and 75-77, respectively;
preferably, according to the IMGT numbering system, the Kabat numbering system, or the Chothia numbering system, the HCDR1, the HCDR2, and the HCDR3 are selected from sequences set forth in SEQ ID NOs: 78-80, 81-83, and 84-86, respectively;
preferably, according to the IMGT numbering system, the Kabat numbering system, or the Chothia numbering system, the HCDR1, the HCDR2, and the HCDR3 are selected from SEQ ID NOs: 88-90, 91-93, and 94-96, respectively.

In some specific embodiments, the CDR1, the CDR2, and/or the CDR3 comprises amino acid sequences having at most 10, 9, 8, 7, 6, 5, 4, 3, 2, or 1 mutation on the HCDR1, the HCDR2, and/or the HCDR3; the mutation is selected from an insertion, a deletion, and/or a substitution, and the substitution is preferably a substitution of conserved amino acids.

In some specific embodiments, the CDR1, the CDR2, and/or the CDR3 comprises sequences having at least 80%, 85%, 90%, 91%, 92%, 93%, 94%, 95%, 96%, 97%, 98%, 99%, or 100% identity to the HCDR1, the HCDR2, and/or the HCDR3, respectively.

In some specific embodiments, the antibody or the antigen-binding fragment comprises a single domain antibody comprising the CDR1, the CDR2, and the CDR3.

In some specific embodiments, the single domain antibody comprises a sequence set forth in any one of SEQ ID NOs: 17-23 and 87; optionally, the single domain antibody comprises a sequence having at least 80%, 85%, 90%, 91%, 92%, 93%, 94%, 95%, 96%, 97%, 98%, 99%, or 100% identity to the sequence set forth in any one of SEQ ID NOs: 17-23 and 87; or the single domain antibody comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 17-23 and 87; the mutation may be selected from an insertion, a deletion, and/or a substitution, and the substitution is preferably a substitution of conserved amino acids.

In some specific embodiments, the single domain antibody comprises an FR region in a VHH domain set forth in any one of SEQ ID NOs: 17-23 and 87; optionally, the single domain antibody comprises a sequence having at least 80%, 85%, 90%, 91%, 92%, 93%, 94%, 95%, 96%, 97%, 98%, 99%, or 100% identity to the FR region in the VHH domain set forth in any one of SEQ ID NOs: 17-23 and 87; or the single domain antibody comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 17-23 and 87; the mutation may be selected from an insertion, a deletion, and/or a substitution, and the substitution is preferably a substitution of conserved amino acids.

In some specific embodiments, the antibody or the antigen-binding fragment is: (1) a chimeric antibody or a fragment thereof, (2) a humanized antibody or a fragment thereof, or (3) a full human antibody or a fragment thereof.

In some specific embodiments, the antibody or the antigen-binding fragment comprises or does not comprise an antibody heavy chain constant region; optionally, the antibody heavy chain constant region may be selected from human, alpaca, mouse, rat, rabbit, and sheep; optionally, the antibody heavy chain constant region may be selected from IgGs IgM, IgA, IgE, and IgD, and the IgG may be selected from IgG1, IgG2, IgG3, and IgG4; optionally, the heavy chain constant region may be selected from an Fc region, a CH3 region, or a complete heavy chain constant region, preferably, the heavy chain constant region is a human Fc region, and more preferably has an amino acid sequence set forth in SEQ ID NO: 1; preferably, the antibody or the antigen-binding fragment is a heavy chain antibody.

In some specific embodiments, the antibody or the antigen-binding fragment is further conjugated to a therapeutic agent or tracer, wherein preferably, the therapeutic agent is selected from a radioisotope, a chemotherapeutic agent, and an immunomodulator, and the tracer is selected from a radiocontrast medium, a paramagnetic ion, a metal, a fluorescent label, a chemiluminescent label, an ultrasound contrast agent, and a photosensitizer.

In some specific embodiments, the VHH domain specifically binds to human, monkey, and/or murine GPC3; preferably, the VHH domain binds to human and/or monkey GPC3 with a KD greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M, or 1.00E-10 M.

In a second aspect, the present disclosure discloses an antibody or an antigen-binding fragment specifically binding to GPC3, wherein the antibody or the antigen-binding fragment comprises a CDR1, a CDR2, and a CDR3 comprising an HCDR1, an HCDR2, and an HCDR3 selected from a VHH domain set forth in any one of SEQ ID NOs:17-23 and 87.

In some specific embodiments, the HCDR1, the HCDR2, and the HCDR3 are determined according to the IMGT numbering system, the Kabat numbering system, or the Chothia numbering system, e.g., selected from Table 1; for example, the HCDR1 is selected from SEQ ID NOs: 24, 27, 30, 33, 36, 39, 42, 45, 48, 51, 54, 57, 60, 63, 66, 69, 72, 75, 78, 81, 84, 88, 91, and 94, the HCDR2 is selected from SEQ ID NOs: 25, 28, 31, 34, 37, 40, 43, 46, 49, 52, 55, 58, 61, 64, 67, 70, 73, 76, 79, 82, 85, 89, 92, and 95, and the HCDR3 is selected from SEQ ID NOs: 26, 29, 32, 35, 38, 41, 44, 47, 50, 53, 56, 59, 62, 65, 68, 71, 74, 77, 80, 83, 86, 90, 93, and 96;
preferably, according to the IMGT numbering system, the Kabat numbering system, or the Chothia numbering system, the HCDR1, the HCDR2, and the HCDR3 are selected from sequences set forth in SEQ ID NOs: 24-26, 27-29, and 30-32, respectively;
preferably, according to the IMGT numbering system, the Kabat numbering system, or the Chothia numbering system, the HCDR1, the HCDR2, and the HCDR3 are selected from sequences set forth in SEQ ID NOs: 33-35, 36-38, and 39-41, respectively;
preferably, according to the IMGT numbering system, the Kabat numbering system, or the Chothia numbering system, the HCDR1, the HCDR2, and the HCDR3 are selected from sequences set forth in SEQ ID NOs: 42-44, 45-47, and 48-50, respectively;
preferably, according to the IMGT numbering system, the Kabat numbering system, or the Chothia numbering system, the HCDR1, the HCDR2, and the HCDR3 are selected from sequences set forth in SEQ ID NOs: 51-53, 54-56, and 57-59, respectively;
preferably, according to the IMGT numbering system, the Kabat numbering system, or the Chothia numbering system, the HCDR1, the HCDR2, and the HCDR3 are selected from sequences set forth in SEQ ID NOs: 60-62, 63-65, and 66-68, respectively;
preferably, according to the IMGT numbering system, the Kabat numbering system, or the Chothia numbering system, the HCDR1, the HCDR2, and the HCDR3 are selected from sequences set forth in SEQ ID NOs: 69-71, 72-74, and 75-77, respectively;
preferably, according to the IMGT numbering system, the Kabat numbering system, or the Chothia numbering system, the HCDR1, the HCDR2, and the HCDR3 are selected from sequences set forth in SEQ ID NOs: 78-80, 81-83, and 84-86, respectively;
preferably, according to the IMGT numbering system, the Kabat numbering system, or the Chothia numbering system, the HCDR1, the HCDR2, and the HCDR3 are selected from SEQ ID NOs: 88-90, 91-93, and 94-96, respectively.

In some specific embodiments, the CDR1, the CDR2, and/or the CDR3 comprises amino acid sequences having at most 10, 9, 8, 7, 6, 5, 4, 3, 2, or 1 mutation on the HCDR1, the HCDR2, and/or the HCDR3; the mutation is selected from an insertion, a deletion, and/or a substitution, and the substitution is preferably a substitution of conserved amino acids.

In some specific embodiments, the CDR1, the CDR2, and/or the CDR3 comprises sequences having at least 80%, 85%, 90%, 91%, 92%, 93%, 94%, 95%, 96%, 97%, 98%, 99%, or 100% identity to the HCDR1, the HCDR2, and/or the HCDR3, respectively.

In a third aspect, the present disclosure further discloses a polypeptide comprising the antibody or the antigen-binding fragment described above, wherein preferably, the polypeptide is further linked to an additional functional molecule, and the additional functional molecule may be selected from one or more of: a signal peptide, a protein tag, an additional antigen-binding molecule, and a cytokine.

In some specific embodiments, the additional antigen-binding molecule specifically binds to an antigen other than GPC3 or binds to a GPC3 epitope different from that of the antibody or the antigen-binding fragment described above;
preferably, the antigen other than GPC3 may be selected from: CD3, preferably CD3E; CD16, preferably CD16A; NKG2D; CD40; 4-1BB; CD137 or CD19; EGFR; EGFRvIII; mesothelin; HER2; EphA2; Her3; EpCAM; MUC1; MUC16; CEA; Claudinl 8.2; a folate receptor; Claudin6; WT1; NY-ESO-1; MAGE3; and ASGPR1 or CDH16;
preferably, the additional antigen-binding molecule is an antibody or antigen-binding fragment; preferably, the polypeptide is a multispecific antigen-binding molecule, and the multispecific antigen-binding molecule may be bispecific, trispecific, or tetraspecific, and more preferably, the multispecific antigen-binding molecule may be divalent, tetravalent, or hexavalent.

In some specific embodiments, the cytokine may be selected from IL2, IL-6, IL-12, IL-15, IL-21, IFN, or TNF-alpha.

In a fourth aspect, the present disclosure further discloses a chimeric antigen receptor (CAR) comprising an extracellular antigen-binding domain, a transmembrane domain, and an intracellular signaling domain, wherein the extracellular antigen-binding domain comprises the antibody or the antigen-binding fragment described above.

In a fifth aspect, the present disclosure further discloses an immune effector cell expressing the chimeric antigen receptor described above, or comprising a nucleic acid fragment encoding the chimeric antigen receptor described above, wherein preferably, the immune effector cell is selected from a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell; and preferably, the immune effector cell is an autoimmune effector cell or an allogeneic immune effector cell.

In a sixth aspect, the present disclosure further discloses an isolated nucleic acid fragment encoding the antibody or the antigen-binding fragment, the polypeptide, or the chimeric antigen receptor described above.

In a seventh aspect, the present disclosure further discloses a vector comprising the nucleic acid fragment described above.

In an eighth aspect, the present disclosure further discloses a host cell comprising the vector described above, wherein preferably, the cell is a prokaryotic cell or a eukaryotic cell, such as a bacteria (Escherichia coli), a fungus (yeast), an insect cell, or a mammalian cell (a CHO cell or a 293T cell).

In a ninth aspect, the present disclosure further discloses a method for preparing the antibody or the antigen-binding fragment or the polypeptide described above, comprising: culturing the cell described above, and isolating an antibody or an antigen-binding fragment expressed by the cell, or isolating a polypeptide expressed by the cell.

In a tenth aspect, the present disclosure further discloses a method for preparing the immune effector cell described above, comprising introducing a nucleic acid fragment encoding the CAR described above into the immune effector cell, wherein optionally, the method further comprises initiating expression of the CAR described above in the immune effector cell.

In an eleventh aspect, the present disclosure further discloses a pharmaceutical composition comprising the antibody or the antigen-binding fragment, the polypeptide, the immune effector cell, the nucleic acid fragment or the vector described above, or a product prepared by the method described above, wherein optionally, the pharmaceutical composition further comprises a pharmaceutically acceptable carrier, diluent or adjuvant; and optionally, the pharmaceutical composition further comprises an additional antineoplastic agent.

In an twelfth aspect, the present disclosure further discloses a method for treating a GPC3-positive tumor or cancer, comprising: administering to a subject an effective amount of the antibody or the antigen-binding fragment, the polypeptide, the immune effector cell, the nucleic acid fragment or the vector described above, a product prepared by the method described above, or the pharmaceutical composition described above, wherein preferably, the GPC3-positive tumor or cancer is selected from liver cancer, gastric cancer, lung cancer, breast cancer, head and neck cancer, bladder cancer, ovarian cancer, cervical cancer, kidney cancer, pancreatic cancer, cervical cancer, liposarcoma, melanoma, adrenal carcinoma, neurilemmoma, malignant fibrous histiocytoma, and esophageal cancer; more preferably, the GPC3-positive tumor or cancer is selected from liver cancer, gastric cancer, lung cancer, and breast cancer.

In a thirteenth aspect, the present disclosure further discloses use of the antibody or the antigen-binding fragment, the polypeptide, the immune effector cell, the nucleic acid fragment or the vector described above, a product prepared by the method described above, or the pharmaceutical composition described above in preparing a medicament for treating a GPC3-positive tumor or cancer, wherein preferably, the GPC3-positive tumor or cancer is selected from liver cancer, gastric cancer, lung cancer, breast cancer, head and neck cancer, bladder cancer, ovarian cancer, cervical cancer, kidney cancer, pancreatic cancer, cervical cancer, liposarcoma, melanoma, adrenal carcinoma, neurilemmoma, malignant fibrous histiocytoma, and esophageal cancer; more preferably, the GPC3-positive tumor or cancer is selected from liver cancer, gastric cancer, lung cancer, and breast cancer.

In a fourteenth aspect, the present disclosure further discloses the antibody or the antigen-binding fragment, the polypeptide, the immune effector cell, the nucleic acid fragment or the vector described above, a product prepared by the method described above, or the pharmaceutical composition described above for use in treating a GPC3-positive tumor or cancer, wherein preferably, the GPC3-positive tumor or cancer is selected from liver cancer, gastric cancer, lung cancer, breast cancer, head and neck cancer, bladder cancer, ovarian cancer, cervical cancer, kidney cancer, pancreatic cancer, cervical cancer, liposarcoma, melanoma, adrenal carcinoma, neurilemmoma, malignant fibrous histiocytoma, and esophageal cancer; more preferably, the GPC3-positive tumor or cancer is selected from liver cancer, gastric cancer, lung cancer, and breast cancer.

In a fifteenth aspect, the present disclosure further discloses a kit comprising the antibody or the antigen-binding fragment, the polypeptide, the immune effector cell, the nucleic acid fragment, the vector or the host cell described above, a product prepared by the method described above, or the pharmaceutical composition described above.

In a sixteenth aspect, the present disclosure further discloses a method for detecting GPC3 expression in a biological sample, comprising contacting the biological sample with the antibody or the antigen-binding fragment described above in a condition allowing formation of a complex between the antibody or the antigen-binding fragment described above and GPC3, wherein preferably, the method further comprises detecting the formation of the complex, and indicating the presence or an expression level of GPC3 in the sample.

In a seventeenth aspect, the present disclosure further discloses use of the antibody or the antigen-binding fragment in preparing a GPC3 assay reagent.

In an eighteenth aspect, the present disclosure further discloses a kit for detecting GPC3 comprising at least the antibody or the antigen-binding fragment.

### Terminology and Definitions

Unless otherwise defined, scientific and technical terms related to the present disclosure have the meanings understood by those of ordinary skills in the art.

Furthermore, unless otherwise stated, terms in the singular form herein shall include the plural form, and vice versa. More specifically, as used in this specification and the appended claims, unless otherwise specified, referents in the singular form following “a” or “an” also include those in the plural form.

The terms “comprise” and “have” are used interchangeably and are intended to indicate that the scheme is open-ended, implying that there may be elements other than those listed in the scheme.

Meanwhile, it should be appreciated that the descriptions “comprise” and “have” as used herein also provide the scheme of “consist of”.

The term “and/or” as used herein includes the meanings of “and”, “or”, and “all or any other combination of elements linked by the term”.

The term “glypican-3 (GPC3)” as used herein refers to a heparan sulfate (HS) glycoprotein. As a member of the heparan sulfate proteoglycans, GPC3 is anchored to cell membrane by glycophosphatidylinositol (GPI). “GPC3” as used herein includes mature or immature full-length wild-type GPC3 proteins or mutants (e.g., point mutation, insertion mutation, or deletion mutation), splice variants, orthologs, and fragments thereof. For example, “GPC3” as used herein may be derived from human, primates such as monkeys (e.g., rhesus monkey and cynomolgus monkey), and rodents such as mice and rats. For example, the human GPC3 amino acid sequence can be found in NCBI: NM_004484.3, and the monkey GPC3 amino acid sequence can be found in NCBI: XP_011739317.1.

The term “specific binding” as used herein means that an antigen-binding molecule (e.g., an antibody) specifically binds to an antigen and substantially identical antigens, generally with a high affinity, but does not bind to unrelated antigens with a high affinity. Affinity is generally reflected in the equilibrium dissociation constant (KD), with a lower KD indicating a higher affinity. In the case of antibodies, a high affinity generally means having a KD of about 10⁻⁷ M or less, about 10⁻⁸ M or less, about 1×10⁻⁹ M or less, about 1×10⁻¹⁰ M or less, 1×10⁻¹¹ M or less, or 1×10⁻¹² M or less. KD is calculated as follows: KD=Kd/Ka, where Kd represents the dissociation rate and Ka represents the association rate. The equilibrium dissociation constant KD can be measured by methods well known in the art, such as surface plasmon resonance (e.g., Biacore) or equilibrium dialysis.

The term “antigen-binding molecule” is used in the broadest sense and refers to a molecule that specifically binds to an antigen. For example, antigen-binding molecules include but are not limited to, antibodies or antibody mimetics. “Antibody mimetic” refers to an organic compound or a binding domain that is capable of specifically binding to an antigen, but is not structurally related to an antibody. For example, antibody mimetics include but are not limited to, affibody, affitin, affilin, a designed ankyrin repeat protein (DARPin), a nucleic acid aptamer, or a Kunitz domain peptide. The term “antibody” herein is used in the broadest sense and refers to a polypeptide or a combination of polypeptides that comprises sufficient sequence from an immunoglobulin heavy chain variable region and/or sufficient sequence from an immunoglobulin light chain variable region and is thus capable of specifically binding to an antigen. The term “antibody” as used herein encompasses various forms and various structures as long as they exhibit the desired antigen-binding activity. The term “antibody” as used herein includes alternative protein scaffolds or artificial scaffolds having grafted complementarity determining regions (CDRs) or CDR derivatives. Such scaffolds include antibody-derived scaffolds comprising mutations introduced to, e.g., stabilize the three-dimensional structure of the antibody, and fully synthetic scaffolds comprising, e.g., biocompatible polymers. See, e.g., Korndorfer et al., 2003, Proteins: Structure, Function, and Bioinformatics, 53(1): 121-129 (2003); and Roque et al., Biotechnol. Prog. 20:639-654 (2004). Such scaffolds may also include non-antibody derived scaffolds, such as scaffold proteins known in the art to be useful for grafting CDRs, including but not limited to tenascin, fibronectin, peptide aptamers, and the like.

The term “antibody” as used herein includes a typical “four-chain antibody”, which belongs to an immunoglobulin consisting of two heavy chains (HCs) and two light chains (LCs). The heavy chain refers to a polypeptide chain consisting of, from the N terminus to the C terminus, a heavy chain variable region (VH), a heavy chain constant region CH1 domain, a hinge region (HR), a heavy chain constant region CH2 domain, a heavy chain constant region CH3 domain; moreover, when the full-length antibody is of IgE isotype, the heavy chain optionally further comprises a heavy chain constant region CH4 domain. The light chain is a polypeptide chain consisting of, from the N terminus to the C terminus, a light chain variable region (VL) and a light chain constant region (CL). The heavy chains are linked to each other and to the light chains through disulfide bonds to form a Y-shaped structure. The heavy chain constant regions of immunoglobulins differ in their amino acid composition and arrangement, and thus in their antigenicity. Accordingly, “immunoglobulin” as used herein can be divided into five classes, or isotypes of immunoglobulins, namely IgM, IgD, IgGs IgA, and IgE, with their corresponding heavy chains being 6, y, a, and E chains, respectively. The Ig of the same class can be divided into different subclasses according to the differences in the amino acid composition of the hinge regions and the number and location of disulfide bonds in the heavy chains. For example, IgG can be divided into IgG1, IgG2, IgG3, and IgG4; and IgA can be divided into IgA1 and IgA2. Light chains are divided into κ or λ chains according to differences in the constant regions. Each of the five classes of Ig may have a κ chain or λ chain.

The term “antibody” herein also includes antibodies that do not comprise a light chain, e.g., heavy-chain antibodies (HCAbs) produced by camelids such as Camelus dromedarius, Camelus bactrianus, Lama glama, Lama guanicoe, Vicugna pacos, and other Camelidae species, as well as immunoglobulin new antigen receptors (IgNARs) found in Chondrichthyes such as shark. The term “heavy chain antibody” as used herein refers to an antibody lacking light chains of conventional antibodies. The term specifically includes but is not limited to homodimeric antibodies comprising VH antigen-binding domains as well as CH2 and CH3 constant domains in the absence of CH1 domains.

The terms “VHH domain”, “nanobody”, and “single domain antibody” (sdAb) herein have the same meaning and are used interchangeably to refer to a single domain antibody consisting of only one heavy chain variable region constructed by cloning a variable region of a heavy chain antibody, which is the smallest antigen-binding fragment with complete functions. Generally, a single domain antibody consisting of only one heavy chain variable region is constructed by obtaining a heavy chain antibody naturally lacking a light chain and a heavy chain constant region 1 (CH1) and then cloning a variable region of an antibody heavy chain.

For further description of “heavy chain antibody”, “single domain antibody”, “VHH domain”, and “nanobody”, reference can be made to: Hamers-Casterman et al., Nature. 1993; 363; 446-8; Reviews of Muyldermans (Reviews in Molecular Biotechnology 74:277-302, 2001); and the following patent applications, which are mentioned as general background: WO 94/04678, WO 95/04079, and WO 96/34103; WO94/25591, WO 99/37681, WO 00/40968, WO 00/43507, WO 00/65057, WO 01/40310, WO 01/44301, EP 1134231, and WO 02/48193; WO97/49805, WO 01/21817, WO 03/035694, WO 03/054016, and WO 03/055527; WO 03/050531; WO 01/90190; WO03/025020; and WO 04/041867, WO 04/041862, WO 04/041865, WO 04/041863, WO 04/062551, WO 05/044858, WO 06/40153, WO 06/079372, WO 06/122786, WO 06/122787, and WO 06/122825, and other conventional technologies mentioned in these applications.

“Antibody” herein may be derived from any animal, including but not limited to human and non-human animals which may be selected from primates, mammals, rodents, and vertebrates, such as Camelidae species, Lama glama, Lama guanicoe, Vicugna pacos, sheep, rabbits, mice, rats, or Chondrichthyes (e.g., shark).

The term “multispecific” means having at least two antigen-binding sites, each of which binds to a different epitope of the same antigen or a different epitope of a different antigen. Thus, terms such as “bispecific”, “trispecific”, and “tetraspecific” refer to the number of different epitopes to which the antibody/antigen-binding molecule can bind.

The term “valency” herein refers to the presence of a specified number of binding sites in an antibody/antigen-binding molecule. Thus, the terms “monovalent”, “divalent”, “tetravalent”, and “hexavalent” refer to the presence of one binding site, two binding sites, four binding sites, and six binding sites, respectively, in the antibody/antigen-binding molecule.

“Full-length antibody”, “complete antibody”, and “intact antibody” herein are used interchangeably and refer to an antibody having a substantially similar structure to a natural antibody.

“Antigen-binding fragment” and “antibody fragment” herein are used interchangeably and do not have the entire structure of an intact antibody, but comprise only a part of the intact antibody or a variant of the part that has the ability to bind to an antigen. For example, “antigen-binding fragment” or “antibody fragment” herein includes but is not limited to Fab, F(ab′)₂, Fab′, Fab′-SH, Fd, Fv, scFv, a diabody, and a single domain antibody.

The term “chimeric antibody” herein refers to an antibody having variable sequences of an immunoglobulin derived from one source organism (e.g., rats, mice, rabbits, or alpacas) and a constant region of an immunoglobulin derived from a different organism (e.g., human). Methods for producing chimeric antibodies are known in the art. See, e.g., Morrison, 1985, Science 229(4719):1202-7; Oi et al., 1986, Bio Techniques 4:214-221; Gillies et al., 1985 J Immunol Methods 125:191-202; which are incorporated herein by reference.

The term “humanized antibody” herein refers to a genetically engineered non-human antibody that has an amino acid sequence modified to increase homology to the sequence of a human antibody. Generally, all or part of the CDR regions of a humanized antibody is derived from a non-human antibody (donor antibody), and all or part of the non-CDR regions (e.g., variable region FRs and/or constant regions) are derived from a human immunoglobulin (acceptor antibody). The humanized antibody generally retains or partially retains the desired properties of the donor antibody, including but not limited to, antigen specificity, affinity, reactivity, the ability to increase the activity of immune cells, the ability to enhance immune response, and the like.

The term “full human antibody” herein refers to an antibody having variable regions in which both the FRs and CDRs are derived from human immunoglobulin species. Furthermore, if the antibody comprises constant regions, the constant regions are also derived from human immunoglobulin species. The full human antibody herein may include amino acid residues that are not encoded by human immunoglobulin species (e.g., mutations introduced by random or site-specific mutagenesis in vitro or by somatic mutations in vivo). However, “full human antibody” herein is not intended to include antibodies in which CDR sequences derived from another mammalian species (e.g., mouse) have been grafted onto human framework sequences.

The term “variable region” herein refers to a region of the heavy or light chain of an antibody involved in the binding of the antibody to an antigen. “Heavy chain variable region” is used interchangeably with “VH” and “HCVR”, and “light chain variable region” is used interchangeably with “VL” and “LCVR”. Heavy and light chain variable domains (VH and VL, respectively) of natural antibodies generally have similar structures, each of which contains four conserved framework regions (FRs) and three hypervariable regions (HVRs). See, e.g., Kindt et al., Kuby Immunology, 6th ed., W. H. Freeman and Co., p. 91 (2007). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region because it can form precise complementarity to an epitope in spatial structures, wherein the heavy chain variable region CDR may be abbreviated as HCDR and the light chain variable region CDR may be abbreviated as LCDR. The term “framework region” or “FR region” is used interchangeably to refer to those amino acid residues of an antibody heavy chain variable region or light chain variable region, other than the CDRs. Generally, a typical antibody variable region consists of 4 FR regions and 3 CDR regions in the following order: FR1-CDR1-FR2-CDR2-FR3-CDR3-FR4.

For further description of the CDRs, reference is made to Kabat et al., J. Biol. 252:6609-6616 (1977); Kabat et al., U.S. Department of Health and Human Services, “Sequences of proteins of immunological interest” (1991); Chothia et al., Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pltickthun, J. Mol. Biol., 309:657-670 (2001). “CDR” as used herein may be labeled and defined in a manner well known in the art, including but not limited to, the Kabat numbering system, the Chothia numbering system, or the IMGT numbering system, using tool sites, including but not limited to, AbRSA site (http://cao.labshare.cn/AbRSA/cdrs.php), abYsis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering system” as used herein generally refers to the immunoglobulin alignment and numbering system proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering system” as used herein generally refers to the immunoglobulin numbering system proposed by Chothia et al., which is a classical rule for identifying CDR region boundaries based on the position of structural loop regions (see, e.g., Chothia & Lesk (1987) J. Mol. Biol. 196:901-917; Chothia et al., (1989) Nature 342:878-883).

The term “IMGT numbering system” herein generally refers to a numbering system based on the international ImMunoGeneTics information system (IMGT) initiated by Lefranc et al., see Lefranc et al., Dev. Comparat. Immunol. 27:55-77, 2003.

For example, Kabat, Chothia, and IMGT are used to determine CDRs of S001-NB150-20(cyno+), S001-NB150-28(cyno+), S001-NB150-56, S001-NB152-1, S001-NB152-101, S001-NB152-72-2, VH001 (SEQ ID NO: 17-23), and VHH-12 (SEQ ID NO: 87). The specific results are shown in Table 1:

TABLE 1
CDR sequences

| Antibody and | Numbering | | | |
|---|---|---|---|---|
| sequence id | system | CDR1 | CDR2 | CDR3 |
| S001-NB150-20(cyno+) | IMGT | GRSFSPYA | ISNSGGST | TGPVKRYSTDFQGGDY |
| (SEQ ID NO: 17) | | (SEQ ID NO: 24) | (SEQ ID NO: 25) | (SEQ ID NO: 26) |
| | Kabat | PYAMG | AISNSGGSTYYADAVKG | PVKRYSTDFQGGDY |
| | | (SEQ ID NO: 27) | (SEQ ID NO: 28) | (SEQ ID NO: 29) |
| | Chothia | GRSFSPY | SNSGGS | PVKRYSTDFQGGDY |
| | | (SEQ ID NO: 30) | (SEQ ID NO: 31) | (SEQ ID NO: 32) |
| S001-NB150-28(cyno+) | IMGT | GSISTYT | ISSGGRT | NSADGLKIGTYYFKGLG |
| (SEQ ID NO: 18) | | (SEQ ID NO: 33) | (SEQ ID NO: 34) | (SEQ ID NO: 35) |
| | Kabat | YTMA | AISSGGRTDYIDSVKG | ADGLKIGTYYFKGLG |
| | | (SEQ ID NO: 36) | (SEQ ID NO: 37) | (SEQ ID NO: 38) |
| | Chothia | GSISTY | SSGGR | ADGLKIGTYYFKGLG |
| | | (SEQ ID NO: 39) | (SEQ ID NO: 40) | (SEQ ID NO: 41) |
| S001-NB150-56 | IMGT | GVDISTYT | ISTTGRS | NSADDLKIGTQYFKGLG |
| (SEQ ID NO: 19) | | (SEQ ID NO: 42) | (SEQ ID NO: 43) | (SEQ ID NO: 44) |
| | Kabat | TYTMA | AISTTGRSIYIDAVQG | ADDLKIGTQYFKGLG |
| | | (SEQ ID NO: 45) | (SEQ ID NO: 46) | (SEQ ID NO: 47) |
| | Chothia | GVDISTY | STTGR | ADDLKIGTQYFKGLG |
| | | (SEQ ID NO: 48) | (SEQ ID NO: 49) | (SEQ ID NO: 50) |
| S001-NB152-1 | IMGT | GRRFSTNV | INWVIGNT | AGRSSYYTSSRREDYDY |
| (SEQ ID NO: 20) | | (SEQ ID NO: 51) | (SEQ ID NO: 52) | (SEQ ID NO: 53) |
| | Kabat | TNVMG | AINWVIGNTNYAESVKG | RSSYYTSSRREDYDY |
| | | (SEQ ID NO: 54) | (SEQ ID NO: 55) | (SEQ ID NO: 56) |
| | Chothia | GRRFSTN | NWVIGN | RSSYYTSSRREDYDY |
| | | (SEQ ID NO: 57) | (SEQ ID NO: 58) | (SEQ ID NO: 59) |
| S001-NB152-101 | IMGT | GRSFGLRA | IGKAGDTT | ATAARWEPPTIT |
| (SEQ ID NO: 21) | | (SEQ ID NO: 60) | (SEQ ID NO: 61) | (SEQ ID NO: 62) |
| | Kabat | LRAMG | AIGKAGDTTYYTDSVKG | AARWEPPTITPGSY |
| | | (SEQ ID NO: 63) | (SEQ ID NO: 64) | (SEQ ID NO: 65) |
| | Chothia | GRSFGLR | GKAGDT | AARWEPPTITPGSY |
| | | (SEQ ID NO: 66) | (SEQ ID NO: 67) | (SEQ ID NO: 68) |
| S001-NB152-72-2 | IMGT | GSILSITA | ITNGGRT | YARRWTGGDRPEYES |
| (SEQ ID NO: 22) | | (SEQ ID NO: 69) | (SEQ ID NO: 70) | (SEQ ID NO: 71) |
| | Kabat | ITAMN | SITNGGRTNYADSVKG | RRWTGGDRPEYES |
| | | (SEQ ID NO: 72) | (SEQ ID NO: 73) | (SEQ ID NO: 74) |
| | Chothia | GSILSIT | TNGGR | RRWTGGDRPEYES |
| | | (SEQ ID NO: 75) | (SEQ ID NO: 76) | (SEQ ID NO: 77) |
| VH001 | IMGT | GFTLDVYA | ITSGDGI | ATLKRTTAGGWP |
| (SEQ ID NO: 23) | | (SEQ ID NO: 78) | (SEQ ID NO: 79) | (SEQ ID NO: 80) |
| | Kabat | VYAIG | YITSGDGIFYADSVKG | LKRTTAGGWPIPGRI |
| | | (SEQ ID NO: 81) | (SEQ ID NO: 82) | (SEQ ID NO: 83) |
| | Chothia | GFTLDVY | TSGDG | LKRTTAGGWPIPGRI |
| | | (SEQ ID NO: 84) | (SEQ ID NO: 85) | (SEQ ID NO: 86) |
| VHH-12 | IMGT | GFTLENYA | ITGGTGTT | ATLKRTTAGGWP |
| (SEQ ID NO: 87) | | (SEQ ID NO: 88) | (SEQ ID NO: 89) | (SEQ ID NO: 90) |
| | Kabat | NYAIG | YITGGTGTTVYADSVKG | LKRTTAGGWPKPGRI |
| | | (SEQ ID NO: 91) | (SEQ ID NO: 92) | (SEQ ID NO: 93) |
| | Chothia | GFTLENY | TGGTGT | LKRTTAGGWPKPGRI |
| | | (SEQ ID NO: 94) | (SEQ ID NO: 95) | (SEQ ID NO: 96) |

The term “heavy chain constant region” as used herein refers to the C-terminal portion of an antibody heavy chain that is not directly involved in the binding of the antibody to an antigen, but exhibits effector functions, such as interaction with an Fc receptor, which has a more conserved amino acid sequence relative to the variable domain of the antibody. The “heavy chain constant region” may be selected from a CH1 domain, a hinge region, a CH2 domain, a CH3 domain, or a variant or fragment thereof. The “heavy chain constant region” includes a “full-length heavy chain constant region” having a structure substantially similar to that of a natural antibody constant region, and a “heavy chain constant region fragment” comprising only a portion of the full-length heavy chain constant region. For example, a typical “full-length antibody heavy chain constant region” consists of the CH1 domain-hinge region-CH2 domain-CH3 domain. When the antibody is IgE, it further comprises a CH4 domain; and when the antibody is a heavy chain antibody, it does not include the CH1 domain. For example, a typical “heavy chain constant region fragment” may be selected from an Fc or CH3 domain.

The term “light chain constant region” as used herein refers to the C-terminal portion of an antibody light chain that is not directly involved in the binding of the antibody to an antigen. The light chain constant region may be selected from a constant κ domain and a constant λ domain.

The term “Fc region” is used herein to define a C-terminal region of an antibody heavy chain comprising at least a portion of the constant region. The Fc region includes a native Fc region and a variant Fc region. For example, a human IgG heavy chain Fc region may extend from Cys226 or Pro230 to the C terminus of a heavy chain. However, an antibody produced by a host cell may undergo post-translational cleavage, and one or more, particularly one or two amino acids may be cut off from the C terminus of the heavy chain. Therefore, an antibody produced by a host cell through expression of a particular nucleic acid molecule encoding a full-length heavy chain may comprise a full-length heavy chain, or may comprise a cleaved variant of the full-length heavy chain. This may be the case when the last two C-terminal amino acids of the heavy chain are glycine (G446) and lysine (K447, numbered according to the Kabat E U index). Therefore, the C-terminal lysine (Lys447), or the C-terminal glycine (Gly446) and lysine (Lys447) in the Fc region may or may not be present.

Typically, an IgG Fc region comprises IgG CH2 and IgG CH3 domains, and optionally, may further comprise a complete or partial hinge region on this basis, but does not comprise a CH1 domain. A “CH2 domain” in an Fc region of human IgG typically extends from the amino acid residue at about position 231 to the amino acid residue at about position 340. In one embodiment, a carbohydrate chain is attached to the CH2 domain. The CH2 domain herein may be a native CH2 domain or a variant CH2 domain. A “CH3 domain” comprises residues at the C terminus of the CH2 domain in the Fc region (i.e., from the amino acid residue at about position 341 to the amino acid residue at about position 447 of the IgG). The CH3 region herein may be a native CH3 domain or a variant CH3 domain (e.g., a CH3 domain having a “knob” introduced in one strand and a “hole” introduced in another strand; see U.S. Pat. No. 5,821,333, which is incorporated herein by reference). As described herein, such variant CH3 domains can be used to promote heterodimerization of two different antibody heavy chains.

Unless otherwise specified herein, amino acid residues in the Fc region or constant region are numbered according to the EU numbering system, also known as the EU index, as described in Kabat et al., Sequences of Proteins of Immunological Interest, 5th Ed. Public Health Service, National Institutes of Health, Bethesda, M D, 1991.

The term “conserved amino acid” herein generally refers to amino acids that belong to the same class or have similar characteristics (e.g., charge, side chain size, hydrophobicity, hydrophilicity, backbone conformation, and rigidity).

For example, the following six groups are examples of amino acids considered to be conservative substitutions for one another:
1) alanine (A), serine (S), and threonine (T);
2) aspartic acid (D) and glutamic acid (E);
3) asparagine (N) and glutamine (Q);
4) arginine (R), lysine (K), and histidine (H);
5) isoleucine (I), leucine (L), methionine (M), and valine (V); and
6) phenylalanine (F), tyrosine (Y), and tryptophan (W).

The term “identity” herein can be obtained by calculating as follows: To determine the percent identity of two amino acid sequences or two nucleic acid sequences, the sequences are compared for optimal alignment (e.g., for optimal alignment, gaps can be introduced in one or both of the first and second amino acid sequences or nucleic acid sequences, or non-homologous sequences can be discarded for comparison). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needlema and Wunsch algorithm ((1970) J. Mol. Biol., 48:444-453; available at www.gcg.com) which has been integrated into the GAP program of the GCG software package, using the Blossom 62 matrix or PAM250 matrix and gap weight of 16, 14, 12, 10, 8, 6, or 4 and length weight of 1, 2, 3, 4, 5, or 6. For another example, the percent identity between two nucleotide acid sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blossom 62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W Miller algorithm ((1989) CABIOS, 4:11-17) which has been incorporated into the ALIGN program (version 2.0).

Additionally or alternatively, the nucleic acid sequences and protein sequences described herein can be further used as “query sequences” to perform searches against public databases to, e.g., identify other family member sequences or related sequences. For example, such searches can be performed using the NBLAST and XBLAST programs (version 2.0) of Altschul et al., (1990) J. Mol. Biol., 215:403-410. BLAST nucleotide searches can be performed using the NBLAST program, with a score of 100 and a word length of 12, to obtain nucleotide sequences homologous to the nucleic acid molecule of the present disclosure. BLAST protein searches can be performed using the XBLAST program, with a score of 50 and a word length of 3, to obtain amino acid sequences homologous to the protein molecule of the present disclosure. To obtain gapped alignment results for comparison purposes, gapped BLAST can be used as described in Altschul et al., (1997) Nucleic Acids Res. 25:3389-3402. When using the BLAST and gapped BLAST programs, the default parameters of the respective programs (e.g., XBLAST and NBLAST) can be used. See www.ncbi.nlm.nih.gov.

The term “chimeric antigen receptor (CAR)” herein refers to an artificial cell surface receptor engineered to express on an immune effector cell and specifically bind to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

The term “nucleic acid” herein includes any compound and/or substance that comprises a polymer of nucleotides. Each nucleotide consists of a base, in particular a purine or pyrimidine base (i.e., cytosine (C), guanine (G), adenine (a), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. The term “nucleic acid molecule” herein encompasses deoxyribonucleic acid (DNA), including, e.g., complementary DNA (cDNA) and genomic DNA; ribonucleic acid (RNA), in particular in the synthetic form of messenger RNA (mRNA), DNA or RNA; and polymers comprising a mixture of two or more of these molecules. The nucleic acid molecule may be linear or cyclic. Furthermore, the term “nucleic acid molecule” includes both sense and antisense strands, as well as single-stranded and double-stranded forms. Moreover, the nucleic acid molecules described herein may contain native or non-native nucleotides. Examples of non-native nucleotides include modified nucleotide bases having derived sugar or phosphate backbone linkages or chemically modified residues. The nucleic acid molecule also encompasses DNA and RNA molecules suitable for use as vectors for direct expression of the antibodies of the present disclosure in vitro and/or in vivo, e.g., in a host or patient. Such DNA (e.g., cDNA) or RNA (e.g., mRNA) vectors may be unmodified or modified. For example, mRNA can be chemically modified to enhance the stability of the RNA vector and/or the expression of the encoded molecule so that the mRNA can be injected into a subject to produce antibodies in vivo (see, e.g., Stadler et al., Nature Medicine 2017, published online, Jun. 12, 2017, doi: 10.1038/nm.4356 or EP 2 101 823 B1). An “isolated” nucleic acid herein refers to a nucleic acid molecule that has been separated from components of its natural environment. The isolated nucleic acid includes a nucleic acid molecule contained in a cell that generally contains the nucleic acid molecule, but the nucleic acid molecule is present extrachromosomally or at a chromosomal location different from its natural chromosomal location.

The term “vector” herein refers to a nucleic acid molecule capable of amplifying another nucleic acid to which it has been linked. The term includes vectors that serve as self-replicating nucleic acid structures as well as vectors integrated into the genome of a host cell into which they have been introduced. Certain vectors are capable of directing the expression of nucleic acids to which they are operably linked. Such vectors are called “expression vectors” herein.

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective, and does not contain additional ingredients having unacceptable toxicity to a subject to which the pharmaceutical composition is administered.

The term “treatment” herein refers to surgical or therapeutic treatment for the purpose of preventing and slowing (reducing) an undesired physiological or pathological change, e.g., cancer and tumor, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission (either partial or complete), whether detectable or undetectable. Subjects in need of treatment include subjects suffering from a disorder or disease as well as subjects susceptible to a disorder or disease or subjects for whom prevention of a disorder or disease is intended. When referring to terms such as slow, moderate, reduce, ameliorate, and alleviate, their meanings also include elimination, disappearance, nonoccurrence, etc.

The term “subject” herein refers to an organism that receives treatment for a particular disease or disorder described herein. For example, “subjects” include mammals, such as human, primates (e.g., monkey), or non-primate mammals, that are receiving a treatment for a disease or disorder.

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate the associated disorder, or to treat, cure, prevent, or alleviate the worsening rate of such disorder. When an active ingredient is administered alone to an individual, a therapeutically effective dose refers to the amount of the ingredient alone. When a combination is used, a therapeutically effective dose refers to amounts of the active ingredients in the combination that produce the therapeutic effect, whether the active ingredients are administered in combination, sequentially, or simultaneously.

The term “cancer” herein refers to or describes a physiological condition in mammals that is typically characterized by unregulated cell growth. This definition includes both benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

The term “EC50” herein refers to the half maximal effective concentration, which includes the antibody concentration that induces a halfway response between the baseline and maximum after a specified exposure time. EC50 essentially represents the antibody concentration at which 50% of the maximal effect is observed, and can be measured by methods known in the art.

The term “GPC3-positive” herein refers to a higher cell expression of GPC3 in cells or tissues than the normal expression level of the corresponding cells or tissues, and “GPC3-positive” can be determined by methods known in the art.

### BRIEF DESCRIPTION OF THE DRAWINGS

FIG. 1A shows binding reactions of control antibodies to human GPC3-His protein by ELISA;

FIG. 1B shows binding reactions of control antibodies to monkey GPC3-His protein by ELISA;

FIG. 1C shows binding reactions of control antibodies to mouse GPC3-His protein by ELISA;

FIG. 2A shows binding reactions of polypeptide GC3pep protein to control antibodies by ELISA;

FIG. 2B shows binding reactions of polypeptide YP7pep protein to control antibodies by ELISA;

FIG. 3A shows FACS result of expression levels of GPC3 in HepG2 cells detected with antibody Tab003;

FIG. 3B shows FACS result of expression levels of GPC3 in HepG2 cells detected with antibody Tab005;

FIG. 4A shows FACS result of expression levels of GPC3 in CHOK1-hGPC3.1C3 cells detected with antibody Tab003;

FIG. 4B shows FACS result of expression levels of GPC3 in CHOK1-hGPC3.2B5 cells detected with antibody Tab003;

FIG. 4C shows FACS result of expression levels of GPC3 in CHOK1-hGPC3.3E9 cells detected with antibody Tab003;

FIG. 5 shows FACS result of expression levels of GPC3 in HEK293T-monkey GPC3 cells detected with antibody Tab003;

FIG. 6A shows alpaca serum antibody titers after immunization with human GPC3 protein by ELISA;

FIG. 6B shows alpaca serum antibody titers after immunization with human GPC3 protein by FACS;

FIGS. 7A and 7B show binding reactions of VHH-hFc to human GPC3-his protein by ELISA;

FIG. 8A shows binding reactions of VHH-hFc to CHO-K1-human GPC3 cells by FACS;

FIG. 8B shows binding reactions of VHH-hFc with CHO-K1 cells by FACS;

FIGS. 9A and 9C show binding reactions of VHH-hFc to HepG2 tumor cells by FACS;

FIGS. 9B and 9D show binding reactions of VHH-hFc to A431 tumor cells by FACS;

FIG. 10 shows binding reactions of VHH-hFc to monkey GPC3-His protein by ELISA;

FIG. 11 shows binding reactions of VHH-hFc to mouse GPC3-his protein by ELISA;

FIG. 12A shows binding reactions of VHH-hFc to HEK293T-monkey GPC3 cells by ELISA;

FIG. 12B shows binding reactions of VHH-hFc to HEK293T cells by ELISA;

FIG. 13A shows binding reactions of VHH-hFc to GC3pep polypeptide protein by ELISA; and

FIG. 13B shows binding reactions of VHH-hFc to YP7pep polypeptide protein by ELISA.

### DETAILED DESCRIPTION

The present disclosure will be further described with reference to specific examples, and the advantages and features of the present disclosure will become more apparent with the description. Experimental procedures without specified conditions in the examples are conducted according to conventional conditions or conditions recommended by the manufacturers. Reagents or instruments without specified manufacturers used herein are conventional products that are commercially available.

The examples are exemplary only and do not limit the scope of the present disclosure in any way. It will be appreciated by those skilled in the art that various changes or substitutions in form and details may be made to the technical schemes of the present disclosure without departing from the spirit and scope of the present disclosure, and that these changes and substitutions shall fall within the scope of the present disclosure.

### Example 1. Preparation of Control Antibodies, Preparation of Human Polypeptides, Identification of Endogenous Cells, and Preparation of Over-Expressing Cells

(A) Preparation of Control Antibodies

VLs and VHs of monoclonal antibodies Y035 and T2-23 which recognize human GPC3 were ligated to human IgG1 Fc in an order from the N terminus to the C terminus, wherein the VH and the VL were ligated by 3 GGGGS linkers to form scFv-human IgG1 Fc (scFv-hFc). The corresponding nucleotide sequences were cloned into a pTT5 vector (purchased from Youbio), and plasmids were prepared according to established standard molecular biological methods. See Sambrook, J., Fritsch, E. F., and Maniatis, T. (1989). Molecular Cloning: A Laboratory Manual, Second Edition (Plainview, New York: Cold Spring Harbor Laboratory Press). For details of Y035 and T2-23, see Patent No. WO2017020812A1, which is incorporated herein by reference. HEK293E cells (purchased from the National Collection of Authenticated Cell Cultures) were transiently transfected with the expression vectors according to PEI (purchased from Polysciences) instructions, cultured at 37° C. for 5 consecutive days using FreeStyle™ 293 (Invitrogen), and centrifuged to remove cell components to give culture supernatants containing antibodies in the form of ScFv-human IgG1 Fc (hFc). The culture supernatants were each loaded on a Protein A chromatography column (the Protein A packing AT Protein A Diamond and the column BXK16/26 were both purchased from Bestchrom), washed with a PBS (phosphate-buffered saline; pH 7.4), then washed with 20 mM PB and 1 M NaCl (pH 7.2), and finally eluted with a citrate buffer at pH 3.4. An Fc-tagged antibody eluted from the Protein A chromatography column was collected, neutralized with 1/10 volume of 1 M Tris at pH 8.0, and dialyzed with PBS at 4° C. overnight. The concentration of the dialyzed antibody was determined using Nanodrop, the purity of the antibody was determined using HPLC-SEC, the endotoxin content of the antibody was determined using an endotoxin assay kit (purchased from Zhanjiang A&C Biological, Ltd.), and finally the antibody was aseptically filtered through a 0.22 μM filter membrane, subpackaged, and stored at −80° C. The antibody in the form of Y035 scFv-hFc and the antibody in the form of T2-23 scFv-hFc were designated Tab003 and Tab005, respectively. Table 2 shows sequences of related control antibodies.

TABLE 2
Sequences of control antibodies

| Sequence | Amino acid sequence |
|---|---|
| hFc | EPKSADKTHTCPPCPAPELLGGPSV |
| (SEQ ID NO: 1) | FLFPPKPKDTLMISRTPEVTCVVVD |
| | VSHEDPEVKFNWYVDGVEVHNAKTK |
| | PREEQYNSTYRVVSVLTVLHQDWLN |
| | GKEYKCKVSNKALPAPIEKTISKAK |
| | GQPREPQVYTLPPSREEMTKNQVSL |
| | TCLVKGFYPSDIAVEWESNGQPENN |
| | YKTTPPVLDSDGSFFLYSKLTVDKS |
| | RWQQGNVFSCSVMHEALHNHYTQKS |
| | LSLSPGK |
| Y035 VH | EVQLVQSGAEVKKPGASVKVSCKAS |
| (SEQ ID NO: 2) | GYTFSDYEMHWVRQAPGQGLEWMGA |
| | IHPGSGDTAYNQRFKGRVTITADKS |
| | TSTAYMELSSLRSEDTAVYYCARFY |
| | SYAYWGQGTLVTVSA |
| Y035 VL | DIVMTQTPLSLPVITGEPASISCRS |
| (SEQ ID NO: 3) | SQSLVHSNGNTYLQWYLQKPGQSPQ |
| | LLIYKVSNRFSGVPDRFSGSGSGTD |
| | FTLKISRVEAEDVGVYYCSQSIYVP |
| | YTFGQGTKLEIKR |
| Y035 scFv-hFc | DIVMTQTPLSLPVITGEPASISCRS |
| (SEQ ID NO: 4) | SQSLVHSNGNTYLQWYLQKPGQSPQ |
| | LLIYKVSNRFSGVPDRFSGSGSGTD |
| | FTLKISRVEAEDVGVYYCSQSIYVP |
| | YTFGQGTKLEIKRGGGGSGGGGSGG |
| | GGSEVQLVQSGAEVKKPGASVKVSC |
| | KASGYTFSDYEMHWVRQAPGQGLEW |
| | MGAIHPGSGDTAYNQRFKGRVTITA |
| | DKSTSTAYMELSSLRSEDTAVYYCA |
| | RFYSYAYWGQGTLVTVSAEPKSADK |
| | THTCPPCPAPELLGGPSVFLFPPKP |
| | KDTLMISRTPEVTCVVVDVSHEDPE |
| | VKFNWYVDGVEVHNAKTKPREEQYN |
| | STYRVVSVLTVLHQDWLNGKEYKCK |
| | VSNKALPAPIEKTISKAKGQPREPQ |
| | VYTLPPSREEMTKNQVSLTCLVKGF |
| | YPSDIAVEWESNGQPENNYKTTPPV |
| | LDSDGSFFLYSKLTVDKSRWQQGNV |
| | FSCSVMHEALHNHYTQKSLSLSPGK |
| T2-23 VH | QVQLQESGGGLVQPGRSLRLSCAAS |
| (SEQ ID NO: 5) | GFTFSSYAMHWVRQAPGKGLEWVSA |
| | ISSSGRSTYYADSVEGRFTISRDNS |
| | KNTLYLQMNSLRAEDTAVYYCAKDR |
| | RGSHADALNVWGQGTLVTVSS |
| T2-23 VL | QSALTQPPSASGSPGQSVTISCTGT |
| (SEQ ID NO: 6) | SSDVGGYNYVSWYQQYPGKAPKLLI |
| | YGNSNRPSGVPDRFSGSKSGTSASL |
| | AITGLQAEDGADYYCQSYDSSLRVV |
| | FGGGTKVTVLG |
| T2-23 scFv-hFc | QSALTQPPSASGSPGQSVTISCTGT |
| (SEQ ID NO: 7) | SSDVGGYNYVSWYQQYPGKAPKLLI |
| | YGNSNRPSGVPDRFSGSKSGTSASL |
| | AITGLQAEDGADYYCQSYDSSLRVV |
| | FGGGTKVTVLGGGGGSGGGGSGGGG |
| | SQVQLQESGGGLVQPGRSLRLSCAA |
| | SGFTFSSYAMHWVRQAPGKGLEWVS |
| | AISSSGRSTYYADSVEGRFTISRDN |
| | SKNTLYLQMNSLRAEDTAVYYCAKD |
| | RRGSHADALNVWGQGTLVTVSSEPK |
| | SADKTHTCPPCPAPELLGGPSVFLF |
| | PPKPKDTLMISRTPEVTCVVVDVSH |
| | EDPEVKFNWYVDGVEVHNAKTKPRE |
| | EQYNSTYRVVSVLTVLHQDWLNGKE |
| | YKCKVSNKALPAPIEKTISKAKGQP |
| | REPQVYTLPPSREEMTKNQVSLTCL |
| | VKGFYPSDIAVEWESNGQPENNYKT |
| | TPPVLDSDGSFFLYSKLTVDKSRWQ |
| | QGNVFSCSVMHEALHNHYTQKSLSL |
| | SPGK |

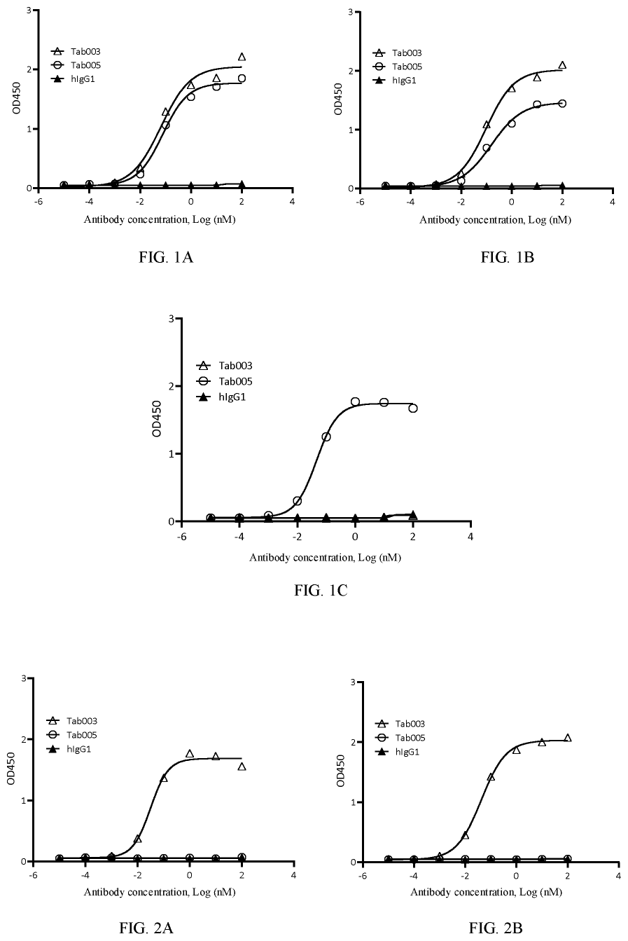

The binding activities of control antibodies to human GPC3-His protein (purchased from Acro, Cat. No. GP3-H52H4), monkey GPC3-His protein (purchased from Acro, Cat. No. GP3-05225), and mouse GPC3-His protein (purchased from Sino Biological, Cat. No. 50989-M08B) are shown in Tables 3-5 and FIGS. 1A-1C, and the IgG subtype control was human IgG1. The results showed that antibodies Tab003 and Tab005 have good binding activity with human GPC3 protein and monkey GPC3 protein; Tab003 does not bind to mouse GPC3 protein; Tab005 binds to the mouse GPC3 protein. The results are consistent with previous reports.

TABLE 3
Binding reactions of control antibodies
to human GPC3-his protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | blank |
| Tab003 | 2.219 | 1.856 | 1.735 | 1.289 | 0.337 | 0.09 | 0.056 | 0.048 |
| Tab005 | 1.85 | 1.706 | 1.534 | 1.06 | 0.231 | 0.073 | 0.068 | 0.049 |
| hIgG1 | 0.072 | 0.05 | 0.048 | 0.05 | 0.048 | 0.048 | 0.047 | 0.049 |

TABLE 4
Binding reactions of control antibodies
to monkey GPC3-his protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| Tab003 | 2.103 | 1.888 | 1.706 | 1.091 | 0.254 | 0.072 | 0.047 | 0.046 |
| Tab005 | 1.446 | 1.431 | 1.103 | 0.694 | 0.131 | 0.057 | 0.044 | 0.046 |
| hIgG1 | 0.056 | 0.047 | 0.044 | 0.045 | 0.044 | 0.044 | 0.048 | 0.046 |

TABLE 5
Binding reactions of control antibodies
to mouse GPC3-his protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| Tab003 | 0.09 | 0.064 | 0.049 | 0.056 | 0.054 | 0.05 | 0.058 | 0.051 |
| Tab005 | 1.672 | 1.761 | 1.77 | 1.252 | 0.303 | 0.086 | 0.05 | 0.052 |
| hIgG1 | 0.105 | 0.055 | 0.05 | 0.05 | 0.052 | 0.051 | 0.049 | 0.057 |

(B) Preparation of Human Polypeptide GC3pep and YP7pep Proteins

GL Biochem was entrusted to manufacture polypeptide GC3pep of human GPC3 (NCBI: NM_004484.3, Ala524-Lys563) and polypeptide YP7pep of human GPC3 (NCBI: NM_004484.3, Asp511-Ser560), with specific sequences shown as follows:

GC3pep:
(SEQ ID NO: 8)
AELAYDLDVDDAPGNSQQATPKDNEISTFHNLGNVHSPLK;
YP7pep:
(SEQ ID NO: 9)
DGMIKVKNQLRFLAELAYDLDVDDAPGNSQQATPKDNEIS
TFHNLGNVHS.

The prepared polypeptides were assayed by ELISA with positive control antibodies recognizing different epitopes. The results are shown in Tables 6 and 7 and FIGS. 2A-2B, suggesting that Tab005 does not bind to polypeptides GC3pep and YP7pep, while the Tab003 binds to polypeptides GC3pep and YP7pep. The results are consistent with previous reports, indicating that polypeptides with binding activity have been prepared.

TABLE 6
Binding reactions of control antibodies
to polypeptide GC3pep protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| Tab003 | 1.554 | 1.722 | 1.769 | 1.366 | 0.373 | 0.088 | 0.056 | 0.049 |
| Tab005 | 0.069 | 0.049 | 0.059 | 0.051 | 0.046 | 0.065 | 0.063 | 0.048 |
| hIgG1 | 0.069 | 0.05 | 0.05 | 0.047 | 0.046 | 0.046 | 0.06 | 0.051 |

TABLE 7
Binding reactions of control antibodies
to polypeptide YP7pep protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| Tab003 | 2.076 | 1.999 | 1.872 | 1.424 | 0.45 | 0.103 | 0.052 | 0.048 |
| Tab005 | 0.057 | 0.052 | 0.046 | 0.05 | 0.048 | 0.049 | 0.045 | 0.046 |
| hIgG1 | 0.056 | 0.048 | 0.048 | 0.056 | 0.045 | 0.047 | 0.058 | 0.052 |

(C) Identification of Cell Lines with Endogenous Expression of GPC3 Protein

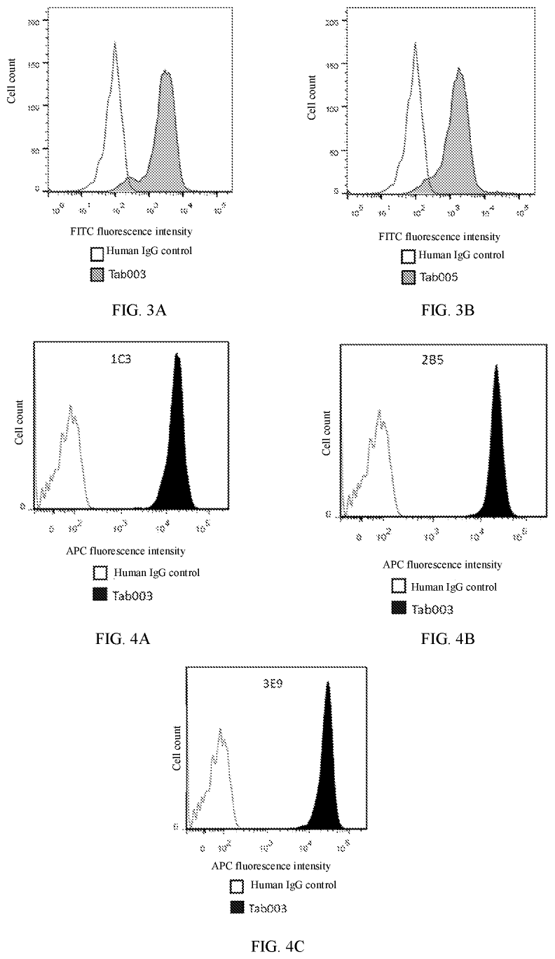

HepG2 cells were expanded in a T-75 cell culture flask to the logarithmic growth phase, and centrifuged to remove the culture supernatant. The cell precipitation was washed twice with PBS. With antibodies Tab003 and Tab005 as primary antibodies and FITC-labeled secondary antibodies (purchased from Invitrogen, Cat. No. A11013), the cells were analyzed by FACS (FACS Canto™, purchased from BD Biosciences). The results are shown in Table 8 and FIGS. 3A-3B, indicating that HepG2 cells binds to both Tab003 and Tab005.

TABLE 8
FACS results of endogenous cell line HepG2

| | | Mean fluorescence density of cells | | |
|---|---|---|---|---|
| | Cells with endogenous | Secondary antibody | | |
| No. | expression | control | Tab003 | Tab005 |
| 1 | HepG2 | 137 | 3647 | 2573 |

(D) Preparation of CHO-Kl Recombinant Cell Line Expressing Human GPC3 Protein

A nucleotide sequence encoding the full-length amino acid sequence of human GPC3 (NCBI: NM_004484.3) was cloned into pcDNA3.1 vector (purchased from Clontech) to prepare a plasmid. After plasmid transfection (Lipofectamine® 3000 Transfection Kit, purchased from Invitrogen, Cat. No. L3000-015), CHO-Kl cells (purchased from the National Collection of Authenticated Cell Cultures) were subjected to a selective culture in a DMEM/F12 medium containing 10% (w/w) fetal bovine serum containing 10 μg/mL of puromycin for 2 weeks, then positive monoclonal cells were sorted onto a 96-well plate on a flow cytometer (FACSAriaII, purchased from BD Biosciences) using antibody Tab003 and donkey anti-human IgG H+L antibody (Jackson, Cat. No. 109605088), and cultured at 5% (v/v) CO₂/37° C. After about 2 weeks, a portion of the monoclonal wells were selected for expansion. The expanded clones were screened by flow cytometry. Monoclonal cell lines with better growth and higher fluorescence intensity were selected for further expansion and cryopreservation in liquid nitrogen. The results of selection are shown in Table 9 and FIGS. 4A-4C, in which the IgG subtype control is a human IgG1 control. Table 9 shows that a series of CHO-Kl monoclonal cell lines with positivity of human GPC3 expression have been prepared. In FIGS. 4A-4C, the abscissa denotes the cell fluorescence intensity, while the ordinate denotes the cell number. The results show that 1C3, 2B5, and 3E9 were cell lines with high-level expression of human GPC3, and 2B5 cells were finally selected for the following examples.

The full-length amino acid sequence of human GPC3 is shown below (SEQ ID NO: 10):

MAGTVRTACLVVAMLLSLDFPGQAQPPPPPPDATCHQVRS
FFQRLQPGLKWVPETPVPGSDLQVCLPKGPTCCSRKMEEK
YQLTARLNMEQLLQSASMELKFLIIQNAAVFQEAFEIVVR
HAKNYTNAMFKNNYPSLTPQAFEFVGEFFTDVSLYILGSD
INVDDMVNELFDSLFPVIYTQLMNPGLPDSALDINECLRG
ARRDLKVFGNFPKLIMTQVSKSLQVTRIFLQALNLGIEVI
NTTDHLKFSKDCGRMLTRMWYCSYCQGLMMVKPCGGYCNV
VMQGCMAGVVEIDKYWREYILSLEELVNGMYRIYDMENVL
LGLFSTIHDSIQYVQKNAGKLTTTIGKLCAHSQQRQYRSA
YYPEDLFIDKKVLKVAHVEHEETLSSRRRELIQKLKSFIS
FYSALPGYICSHSPVAENDTLCWNGQELVERYSQKAARNG
MKNQFNLHELKMKGPEPVVSQIIDKLKHINQLLRTMSMPK
GRVLDKNLDEEGFESGDCGDDEDECIGGSGDGMIKVKNQL
RFLAELAYDLDVDDAPGNSQQATPKDNEISTFHNLGNVHS
PLKLLTSMAISVVCFFFLVH.

TABLE 9
CHO-K1 recombinant cell lines expressing human GPC3 protein
by FACS

| | | Mean fluorescence | |
|---|---|---|---|
| | | density of cells | |
| | | IgG subtype | GPC3 |
| No. | Clone No. of transfected cell | control | antibody |
| 1 | CHOK1-hGPC3.1C3 | 19.3 | 14300 |
| 2 | CHOK1-hGPC3.2B5 | 19.3 | 18900 |
| 3 | CHOK1-hGPC3.3E9 | 19.3 | 26700 |

(E) Preparation of Recombinant HEK293T Cell Lines Expressing Monkey GPC3 Protein

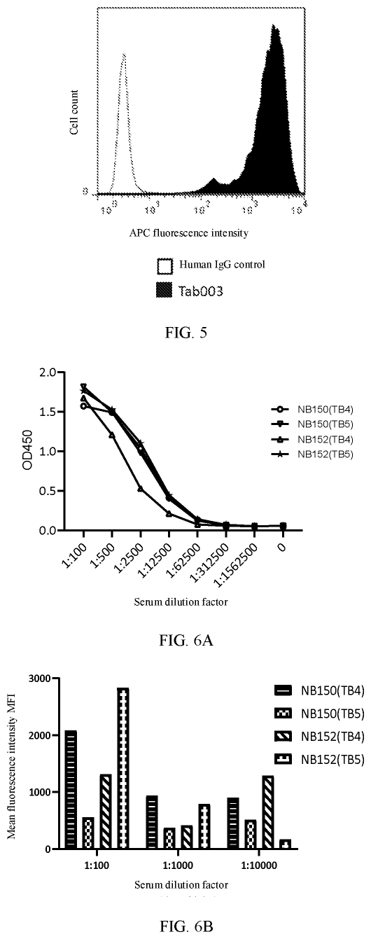

A nucleotide sequence encoding the full-length amino acid sequence of monkey GPC3 (NCBI: XP_011739317.1, SEQ ID NO: 11) was cloned into pcDNA3.1 vector (purchased from Thermofisher Scientific) to give a plasmid. After plasmid transfection with FuGENE® HD (Promega, Cat. No. E2311), HEK293T cell lines were subjected to selective culture in a DMEM medium containing 10% (w/w) fetal bovine serum containing 10 μg/mL of puromycin for 2 weeks, then enriched positive monoclonal cells were sorted onto a 96-well plate on a flow cytometer (FACSAriaII, purchased from BD Biosciences) using antibody Tab003 and donkey anti-human IgG H+L antibody (Jackson, Cat. No. 109605088), cultured at 5% (v/v) CO₂/37° C., and expanded after about 1 week. The expanded cells were analyzed by flow cytometry, and cell lines with better growth and higher fluorescence intensity were selected for further expansion and cryopreservation in liquid nitrogen. The expression levels are shown in FIG. 5, indicating that after puromycin selective culture, HEK293T-monkey-GPC3 has a single positive peak and can thus be used for detecting the cross-activity of antibodies.

The full-length amino acid sequence of monkey GPC3 is shown below (SEQ ID NO: 11):

MAGTVRTACLVVAMLLSLDFPGQAQPPPPPPDATCHQVRS
FFQRLQPGLKWVPETPVPGSDLQVCLPKGPTCCSRKMEEK
YQLTARLNMEQLLQSASMELKFLIIQNAAVFQEAFEIVVR
HAKNYTNAMFKNNYPSLTPQAFEFVGEFFTDVSLYILGSD
INVDDMVNELFDSLFPVIYTQLMNPGLPDSALDINECLRG
ARRDLKVFGNFPKLIMTQVSKSLQVTRIFLQALNLGIEVI
NTTDHLKFSKDCGRMLTRMWYCSYCQGLMMVKPCGGYCNV
VMQGCMAGVVEIDKYWREYILSLEELVNGMYRIYDMENVL
LGLFSTIHDSIQYVQKNAGKLTTTIGKLCAHSQQRQYRSA
YYPEDLFIDKKVLKVAHVEHEETLSSRRRELIQKLKSFIS
FYSALPGYICSHSPVAENDTLCWNGQELVERYSQKAARNG
MKNQFNLHELKMKGPEPVVSQIIDKLKHINQLLRTMSVPK
GRVLDKNLDEEGFESGDCGDDEDECIGGSGDGMMKVKNQL
RFLAELAYDLDVDDVPGNNQQATPKDNEISTFHNLGNVHS
PLKLLTSMAISVVCFFFLVH.

### Example 2. Preparation of Single Domain Antibody VHH Against GPC3

(A) Alpaca Immunization and Serum Titer Assay

GL Biochem was entrusted to manufacture polypeptide GC3pep coupled to a carrier protein (keyhole limpet hemocyanin, or KLH), i.e., polypeptide GC3pep-KLH (AELAYDLDVDDAPGNSQQATPKDNEISTFHNLGNVHSPLKC-KLH).

Two alpacas (Alpaca, numbered NB150 and NB152) were immunized with human GPC3 (G1n25-His559)-His protein (purchased from Acro, Cat. No. GP3-H52H4) and polypeptide GC3pep-KLH. For the first immunization, human GPC3-His protein was emulsified with Freund's complete adjuvant and then injected subcutaneously at multiple sites, with each alpaca receiving 500 μg of human GPC3-His protein. For the first three booster immunizations, human GPC3-His protein was emulsified with Freund's incomplete adjuvant and then injected subcutaneously at multiple sites, with each alpaca receiving 250 μg of human GPC3-His protein. For the fourth booster immunization, polypeptide GC3pep-KLH was emulsified with Freund's incomplete adjuvant and then injected subcutaneously at multiple sites, with each alpaca receiving 250 μg of polypeptide GC3pep-KLH. The interval between the first immunization and the first booster immunization was 3 weeks, the interval between the first booster immunization and the second booster immunization was 3 weeks, the interval between the second booster immunization and the third booster immunization was 3 weeks, and the interval between the third booster immunization and the fourth booster immunization was 2 months.

Blood samples were collected 1 week after each booster immunization, and the antibody titer and specificity of human GPC3-His in serum were determined by ELISA and FACS, with results shown in FIGS. 6A-6B and Table 10. The results show that the serum of alpacas immunized with human GPC3-His and polypeptide GC3pep-KLH exhibited different degrees of binding to the immunogen, showing antigen-antibody reaction, wherein the maximum dilution factor for ELISA was around 12500. Among them, the blank control was 1% (w/w) BSA, and the Batch referred to alpaca sera on day seven after the fourth (TB4) or fifth (TB5) immunizations. Data in the table are OD450 nm values.

Table 10. Antibody titers in alpaca serum after immunization with human GPC3-His protein by ELISA

TABLE 10
Antibody titers in alpaca serum after immunization with
human GPC3-His protein by ELISA
OD450 nm

| Batch | NB150 | NB150 | NB152 | NB152 |
|---|---|---|---|---|
| Serum dilution factor | (TB4) | (TB5) | (TB4) | (TB5) |
| 1:100 | 1.57 | 1.82 | 1.67 | 1.76 |
| 1:500 | 1.49 | 1.50 | 1.21 | 1.53 |
| 1:2500 | 1.04 | 0.98 | 0.53 | 1.10 |
| 1:12500 | 0.41 | 0.40 | 0.21 | 0.45 |
| 1:62500 | 0.13 | 0.12 | 0.07 | 0.14 |
| 1:312500 | 0.07 | 0.07 | 0.06 | 0.07 |
| 1:1562500 | 0.05 | 0.05 | 0.05 | 0.05 |
| Blank control | 0.06 | 0.06 | 0.06 | 0.06 |

(B) Construction of Phage Library and Panning of GPC3 Nanobody

One week after the fifth (TB5) immunization, 100 mL of alpaca peripheral blood was collected. PBMCs were isolated using a lymph separation buffer, and total RNA was extracted using an RNAiso Plus reagent. The extracted RNA was reversely transcribed into cDNA using PrimeScript™ II 1st Strand cDNA Synthesis Kit (purchased from Takara, Cat. No. 6210A). Variable region nucleic acid fragments encoding heavy chain antibodies were amplified by nested PCR:

First PCR:

upstream primer (SEQ ID NO: 12):
CTTGGTGGTCCTGGCTGC;
downstream primer (SEQ ID NO: 13):
GGTACGTGCTGTTGAACTGTTCC.

Second PCR:

taking the product of the first PCR as the template,

upstream primer (SEQ ID NO: 14):
CATGCCATGACTGTGGCCCAGGCGGCCCAGKTGCAGCTCG
TGGAGTC;
downstream primer-1 (SEQ ID NO: 15):
CATGCCATGACTCGCGGCCGGCCTGGCCATGGGGGTCTTC
GCTGTGGTGCG;
downstream primer-2 (SEQ ID NO: 16):
CATGCCATGACTCGCGGCCGGCCTGGCCGTCTTGTGGTTT
TGGTGTCTTGGG

The nucleic acid fragments of the single domain antibody of interest were recovered and then cloned into a vector pcomb3XSS for phage display by using restriction endonuclease SfiI. Competent E. coli cells TG1 were electrotransformed with the product, and a single domain antibody phage display library against GPC3 was constructed and verified. The calculated size of the library was 3.4×10⁹ by gradient dilution plating. To determine the insertion rate of the library, 48 clones were randomly selected for colony PCR. The results show that the insertion rate was 100%.

(C) Panning of Single Domain Antibodies Against GPC3

Human GPC3-Llama-Fc protein (purchased from Acro, Cat. No. GP3-H5257) was diluted with a carbonate buffer (pH 9.6) to a final concentration of 5 μg/mL. The dilutions were added to a plate at 100 μL/well, with each protein coating 8 wells at 4° C. overnight. The coating buffer was discarded, and the plate was washed 3 times with PBS. 300 μL, of 3% BSA-PBS blocking solution was added to each well for blocking at 37° C. for 1 h. After washing the plate 3 times with PBS, 100 μL, of phage library was added for incubation at 37° C. for 1 h. Unbound phage was removed by pipetting before washing the plate 6 times with PBST and twice with PBS. 100 IA of Gly-HCl eluent was added, and the plate was incubated at 37° C. for 8 min. Specifically-bound phages were removed. The washings were then transferred into a 1.5-mL sterile centrifuge tube, and quickly neutralized with 10 μL, of Tris-HCl neutralization buffer. 10 μL, of the neutralized washing was serially diluted to determine the titer, and the recovery was calculated. The remaining washings were mixed for amplification and purification, and were then used for the next affinity panning.

From the plate of the first panning, 192 monoclones were randomly picked with a sterilized toothpick, and inoculated into 1 mL of 2×YT-A for shaking culture at 220 r/min for 8 h at 37° C. To 100 μL, of the culture, M13K07 phage was added in a ratio of cell:phage=1:20. The mixture was let stand for 15 min at 37° C. and shaken at 220 r/min for 45 min. Another 300 μL, of 2×YT-AK was added for culture overnight at 30° C. under vigorous shaking. The next day, the culture was centrifuged at 12000 rpm for 2 min before the supernatant was subjected to monoclonal identification by ELISA.

Human GPC3 protein and GC3pep polypeptide were diluted with a carbonate buffer (pH 9.6) to a final concentration of 2 μg/mL. The dilutions were added into a plate at 100 μL/well, and incubated at 4° C. overnight. The coating buffer was discarded, and the plate was washed 3 times with PBST. 300 μL, of 5% skimmed milk was added to each well for blocking at 37° C. for 1 h. After the plate was washed 3 times with PBST, 50 μL of phage culture supernatant and 50 μL of 5% skimmed milk were added to each well for incubation at 37° C. for 1 h. After the plate was washed 5 times with PBST, a horseradish peroxidase-labeled anti-M13 antibody (1:10000 diluted with PBS) was added at 100 μL/well for reaction at 37° C. for 1 h. The plate was washed 6 times with PBST. TMB substrate was added at 100 μL/well for chromogenesis at 37° C. for 7 min. A stop solution was added at 50 μL/well to stop the reaction, so as to measure the optical density at a wavelength of 450 nm. Human GPC3 protein/GC3pep polypeptide dual positive clones were selected and sent to Chengdu Tsingke Biotech Co., Ltd. for sequencing.

The sequencing results were analyzed, and an evolutionary tree was constructed according to VHH protein coding sequences. According to sequence similarity, sequences that were close to each other on the evolutionary tree were eliminated for VHH-hFc production and identification. VHH sequences for production and identification are shown below, and CDR regions of the VHH sequences were determined by the IMGT website (http://www.imgt.org/3Dstructure-DB/cgi/DomainGapAlign.cgi#results), the abYsis website (www.abysis.org/abysis/sequence_input/key_annotation/key_annotation.cgi), and the AbRSA website (http://cao.labshare.cn/AbRSA/cdrs.php). See Table 1 for details.

>S001-NB150-20(cyno+)
(SEQ ID NO: 17)
QVQLVESGGGLVQAGGSLKLSCAASGRSFSPYAMGWFRQA
PGKDREFVAAISNSGGSTYYADAVKGRFSISRDNAKNTVY
LQMNNLEPEDTAVYYCTGPVKRYSTDFQGGDYWGQGTQVT
VSS
>S001-NB150-28(cyno+)
(SEQ ID NO: 18)
QVQLVESGGGLVQPGGSLRLSCAASGSISTYTMAWYRQAP
GEQRESVAAISSGGRTDYIDSVKGRFTISRDNAKNMVYLQ
MNSLKPEDTAVYYCNSADGLKIGTYYFKGLGWGQGTQVTV
SA
>S001-NB150-56
(SEQ ID NO: 19)
QVQLVESGGGLVRPGGSLRVSCAASGVDISTYTMAWYRQA
PGEQRESVAAISTTGRSIYIDAVQGRFTMSRDNAKNTVYL
QMNNLKPEDTAVYYCNSADDLKIGTQYFKGLGWGQGTQVT
VSS
>S001-NB152-1
(SEQ ID NO: 20)
QVQLVESGGGLVQAGGSLSLSCAASGRRFSTNVMGWFRQA
PGKEREFLAAINWVIGNTNYAESVKGRFTISRDNAKETVY
LQMDNLKVEDTAVYYCAGRSSYYTSSRREDYDYWGQGTQV
TVSS
>S001-NB152-101
(SEQ ID NO: 21)
QVQLVESGGGLVQAGASLRLSCLGSGRSFGLRAMGWFRQA
PGKELEFVAAIGKAGDTTYYTDSVKGRFTISRDNVKNAVY
LQMNSLKPEDTAVYVCATAARWEPPTITPGSYRGPGTQVT
VSS
>S001-NB152-72-2
(SEQ ID NO: 22)
QVQLVESGGGLVQAGGSLRLSCAASGSILSITAMNWHRQA
PGKERELVASITNGGRTNYADSVKGRFTISRDRAKGTLYL
QMNNLKPEDTAVYYCYARRWTGGDRPEYESWGQGTQVTVS
S
>VH001
(SEQ ID NO: 23)
QLQLVESGGGLVQPGGSLRLSCAASGFTLDVYAIGWFRQA
PGKEREGVSYITSGDGIFYADSVKGRFTISRDNAKNTVYL
QMNSLKPEDTAVYFCATLKRTTAGGWPIPGRIGGQGTQVT
VSS
>VHH-12
(SEQ ID NO: 87)
QVQLVESGGGLVQAGGSLRLSCAASGFTLENYAIGWFRQA
PGKEREGVSYITGGTGTTVYADSVKGRFTISRDNTKNTVY
LQMNSLKPEDTAVYFCATLKRTTAGGWPKPGRIGGQGTQV
TVSS

### Example 3. Production of VHH-hFc

The target VHH sequence was recombined with an expression vector of human IgG1 Fc to give a recombinant plasmid. For specific plasmid construction, transfection and purification procedures, refer to Example 1, section (A). The purified VHH-hFc was analyzed for protein concentration, purity, endotoxicity (Lonza kit), and the like, with results shown in Table 11. The results show that the final antibody product has a high purity, and the endotoxin concentration is no more than 1.0 EU/mg.

TABLE 11
Quality control over purified VHH-hFc

| | Antibody | | |
|---|---|---|---|
| | concentration | Purity of antibody | Endotoxin, |
| Antibody | (mg/mL) | (SEC, 280 nm) % | EU/mg |
| S001-NB150-20(cyno+) | 2.08 | 99.396 | <1 |
| S001-NB150-28(cyno+) | 2.78 | 98.96 | <1 |
| S001-NB150-56 | 2.45 | 98.306 | <1 |
| S001-NB152-1 | 0.42 | 92.064 | <1 |
| S001-NB152-101 | 0.88 | 99.005 | <1 |
| S001-NB152-72-2 | 1.42 | 99.376 | <1 |
| VH001 | 3.49 | 99.905 | <1 |
| VHH-12 | 3.03 | 99.842 | <1 |

### Example 4. Identification of VHH-hFc Antibodies

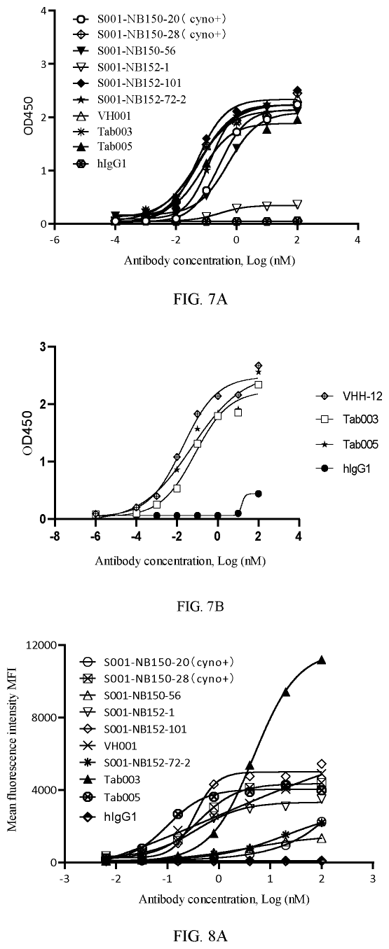

(A) Binding of VHH-hFc to human GPC3 protein by enzyme-linked immunosorbent assay (ELISA) Human GPC3 protein was diluted with PBS to a final concentration of 1 μg/mL, and the dilution was added to a 96-well ELISA plate at 50 μL/well. The plate was sealed with a plastic film and incubated at 4° C. overnight, and washed twice with PBS the next day. A blocking solution [PBS+2% (w/w) BSA] was added for blocking at room temperature for 2 h. The blocking solution was discarded, and VHH-hFc, positive control antibody and negative control antibody serially diluted from 100 nM were added at 50 μL/well. After incubation for 2 h at 37° C., the plate was washed 3 times with PBS. Horseradish peroxidase (HRP)-labeled secondary antibody (purchased from Merck, Cat. No. AP113P) was added for incubation at 37° C. for 1 h. The plate was washed 5 times with PBS. TMB substrate was added at 50 μL/well for incubation at room temperature for 10 min before a stop solution (1.0 M HCl) was added at 50 μL/well. OD450 nm values were measured on an ELISA plate reader (Multimode Plate Reader, EnSight, purchased from Perkin Elmer). The binding activities of VHH-hFc to human GPC3 protein are shown in FIGS. 7A-7B, Table 12-1, and Table 12-2. Antibody S001-NB152-1 binds weakly to human GPC3 protein while the other seven VHH-hFc antibodies bind well to human GPC3 protein. The IgG control was hIgG1, and the data in the table are OD450 nm values.

TABLE 12-1
Binding reactions of VHH-hFc to human GPC3 protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| S001-NB150-20(cyno+) | 2.238 | 1.958 | 1.72 | 0.624 | 0.095 | 0.051 | 0.048 | 0.051 |
| S001-NB150-28(cyno+) | 2.449 | 2.025 | 1.936 | 1.481 | 0.396 | 0.122 | 0.05 | 0.048 |
| S001-NB150-56 | 2.109 | 1.894 | 1.418 | 0.521 | 0.21 | 0.207 | 0.149 | 0.047 |
| S001-NB152-1 | 0.354 | 0.325 | 0.282 | 0.127 | 0.055 | 0.053 | 0.046 | 0.047 |
| S001-NB152-101 | 2.504 | 2.212 | 2.11 | 1.6 | 0.406 | 0.096 | 0.05 | 0.047 |
| S001-NB152-72-2 | 2.197 | 2.245 | 2.022 | 1.007 | 0.194 | 0.109 | 0.084 | 0.085 |
| VH001 | 2.355 | 2.08 | 1.948 | 1.471 | 0.495 | 0.241 | 0.051 | 0.049 |
| Tab003 | 2.199 | 2.058 | 1.89 | 1.443 | 0.506 | 0.268 | 0.107 | 0.048 |
| Tab005 | 1.957 | 1.774 | 1.746 | 1.139 | 0.365 | 0.23 | 0.1 | 0.048 |
| hIgG1 | 0.053 | 0.045 | 0.045 | 0.045 | 0.044 | 0.046 | 0.052 | 0.049 |

TABLE 12-2
Binding reactions of VHH-hFc to human GPC3 protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| VHH-12 | 2.67 | 2.16 | 2.14 | 1.83 | 1.08 | 0.400 | 0.200 | 0.090 |
| Tab003 | 2.34 | 1.85 | 1.790 | 1.310 | 0.530 | 0.250 | 0.100 | 0.080 |
| Tab005 | 2.560 | 1.910 | 1.840 | 1.570 | 0.860 | 0.400 | 0.180 | 0.080 |
| hIgG1 | 0.440 | 0.100 | 0.060 | 0.060 | 0.060 | 0.060 | 0.060 | 0.070 |

(B) Binding of Antibodies to Cells with GPC3 Expression and Cells without GPC3 Expression (Negative Cells) by Flow Cytometry (FACS)

The desired cells were expanded to the logarithmic growth phase in a T-75 cell culture flask, the medium was removed by pipetting, and the cells were washed twice with a PBS buffer, and digested with trypsin. Then a complete medium was added to stop the digestion, and the cells were re-suspended by pipetting to give single cell suspensions. The cells were counted and centrifuged, and resuspended in an FACS buffer (PBS+2% fetal bovine serum) to 2×10⁶ cells/mL. The cell resuspension was added to a 96-well FACS plate at 50 μL/well, and VHH-hFc, the positive control antibody or the negative control antibody was added at 50 μL/well for incubation at 4° C. for 1 h. After the plate was centrifuged and washed 3 times with a PBS buffer, the secondary antibody anti-hIgG(H+L) Alexa 647 (purchased from Jackson, Cat. No. 109-605-088) was added at 50 μL/well and the plate was incubated on ice for 1 h. After the plate was centrifuged and washed 3 times with a PBS buffer, 100 μL of the resultant solution was analyzed by FACS (FACS Canto™, purchased from BD Biosciences). Data analysis was performed by software (FlowJo) to give the mean fluorescence intensity (MFI) of the cells. Then, analysis was performed by software (GraphPad Prism8), the data were fitted, and the EC50 values were calculated.

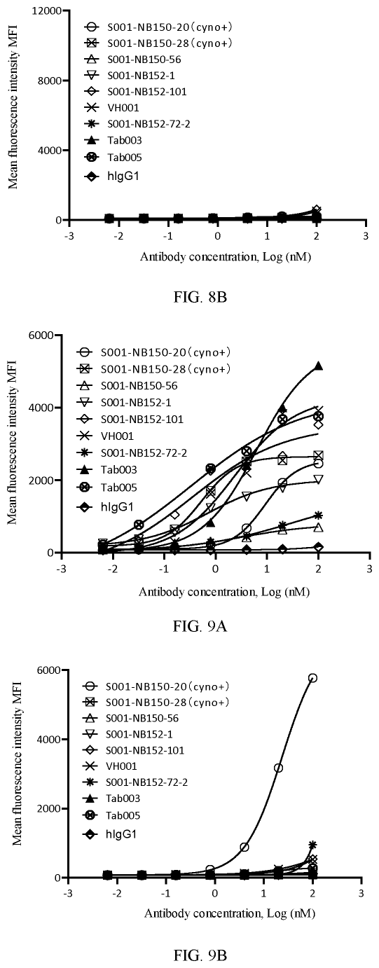

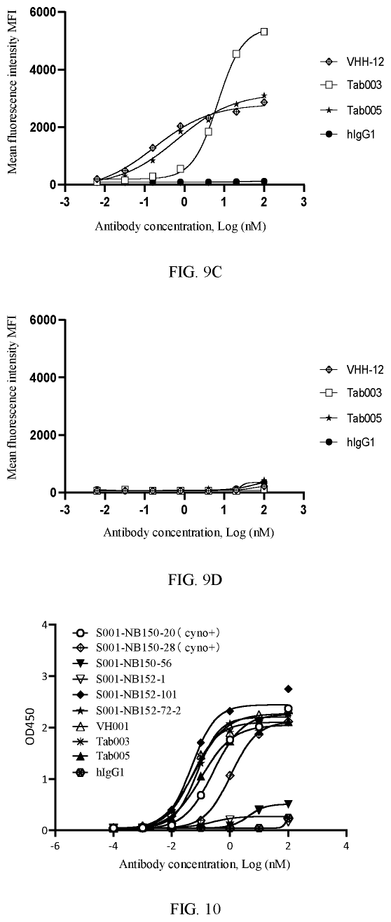

The analysis results are shown in Table 13, Table 14, and FIGS. 8A-8B and 9A-9D (NB represents No binding). Table 13 and FIGS. 8A-8B show that VHH-hFc binds to CHO-Kl-human GPC3 cells but does not bind to CHO-Kl cells; Table 14-1, Table 14-2, and FIGS. 9A-9D show that VHH-hFc binds to HepG2 cells, and the other antibodies than antibody S001-NB150-20(cyno+) do not bind to A431 cells.

TABLE 13
Binding reactions of VHH-hFc to CHO-K1-human GPC3 cells and
CHO-K1 cells by FACS

| | CHO-K1-human GPC3 | | CHO-K1 | |
|---|---|---|---|---|
| | Maximum | | Maximum | |
| | mean | | mean | |
| | fluorescence | | fluorescence | |
| | intensity | EC50 | intensity | EC50 |
| Antibody | Max MFI | (nM) | Max MFI | (nM) |
| S001-NB150-20(cyno+) | 2245 | Weak | 146 | NB |
| S001-NB150-28(cyno+) | 4391 | 0.40 | 122 | NB |
| S001-NB150-56 | 1332 | 3.74 | 137 | NB |
| S001-NB152-1 | 3510 | 0.37 | 141 | NB |
| S001-NB152-101 | 5439 | 0.34 | 610 | NB |
| VH001 | 4897 | 0.97 | 510 | NB |
| S001-NB152-72-2 | 2184 | 21.65 | 197 | NB |
| Tab003 | 11199 | 4.79 | 96 | NB |
| Tab005 | 3982 | 0.10 | 212 | NB |
| hIgG1 | 88 | NB | 107 | NB |

TABLE 14-1
Binding reactions of VHH-hFc to HepG2 cells and A431 cells by FACS

| | HepG2 | | A431 | |
|---|---|---|---|---|
| | Maximum mean | | Maximum mean | |
| | fluorescence | | fluorescence | |
| | intensity | EC50 | intensity | EC50 |
| Antibody | Max MFI | (nM) | Max MFI | (nM) |
| S001-NB150-20(cyno+) | 2460 | 9.22 | 5768 | 23.49 |
| S001-NB150-28(cyno+) | 2678 | 0.53 | 97 | NB |
| S001-NB150-56 | 699 | 3.19 | 123 | NE |
| S001-NB152-1 | 2018 | 0.71 | 151 | NB |
| S001-NB152-101 | 3532 | 0.35 | 540 | NB |
| VH001 | 3506 | 2.39 | 529 | NB |
| S001-NB152-72-2 | 1028 | 31.72 | 952 | NB |
| Tab003 | 5166 | 6.35 | 83 | NB |
| Tab005 | 3759 | 0.33 | 277 | NB |
| hIgG1 | 155 | NB | 114 | NB |

TABLE 14-2
Binding reactions of VHH-hFc to HepG2 cells and A431 cells by FACS

| | HepG2 | | A431 | |
|---|---|---|---|---|
| | Maximum mean | | Maximum mean | |
| | fluorescence intensity | EC50 | fluorescence intensity | EC50 |
| Antibody | Max MFI | (nM) | Max MFI | (nM) |
| VHH-12 | 2864 | 0.18 | 217 | NB |
| Tab003 | 5316 | 6.81 | 123 | NB |
| Tab005 | 3103 | 0.65 | 433 | NB |
| hIgG1 | 124 | NB | 353 | NB |

### Example 5. Cross-Binding Activity of VHH-hFc

(A) Binding of VHH-hFc to monkey GPC3 protein and mouse GPC3 protein by ELISA Monkey GPC3-His protein (purchased from Acro, Cat. No. GP3-05225) was subjected to ELISA and data analysis according to the method described in Example 4, section (A). The ELISA results for VHH-hFc and monkey GPC3 protein are shown in FIG. 10 and Table 15. The results show that antibody S001-NB150-56 has weak binding capacity to monkey GPC3 protein, the S001-NB152-1 antibody does not bind to monkey GPC3 protein, and the other VHH-hFc antibodies bind well to monkey GPC3 protein. The IgG control was hIgG1, and the data in the table are OD450 nm values.

TABLE 15
Binding reactions of VHH-hFc to monkey GPC3 protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| S001-NB150-20(cyno+) | 2.37 | 2.013 | 1.764 | 0.684 | 0.097 | 0.049 | 0.046 | 0.047 |
| S001-NB150-28(cyno+) | 2.118 | 1.868 | 1.065 | 0.195 | 0.056 | 0.052 | 0.043 | 0.047 |
| S001-NB150-56 | 0.508 | 0.406 | 0.092 | 0.049 | 0.044 | 0.045 | 0.043 | 0.045 |
| S001-NB152-1 | 0.166 | 0.398 | 0.203 | 0.12 | 0.06 | 0.045 | 0.045 | 0.049 |
| S001-NB152-101 | 2.753 | 2.137 | 2.32 | 1.706 | 0.4 | 0.092 | 0.051 | 0.047 |
| S001-NB152-72-2 | 2.254 | 2.159 | 2.074 | 1.309 | 0.236 | 0.063 | 0.046 | 0.037 |
| VH001 | 2.347 | 2.243 | 1.906 | 1.465 | 0.351 | 0.086 | 0.048 | 0.047 |
| Tab003 | 2.283 | 1.947 | 1.928 | 1.431 | 0.364 | 0.077 | 0.048 | 0.046 |
| Tab005 | 2.118 | 1.929 | 1.739 | 1.058 | 0.233 | 0.066 | 0.046 | 0.048 |
| hIgG1 | 0.237 | 0.045 | 0.045 | 0.051 | 0.044 | 0.045 | 0.044 | 0.047 |

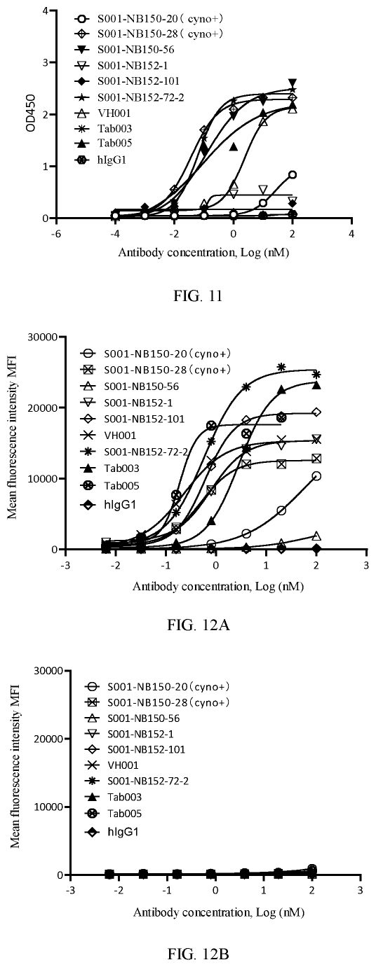

Mouse GPC3-his protein (purchased from Sino Biological, Cat. No. 50989-M08B) was subjected to ELISA and data analysis according to the method described in Example 4, section (A). The ELISA results for VHH-hFc and mouse GPC3 protein are shown in FIG. 11 and Table 16. The results show that antibody S001-NB152-1 has weak binding capacity to mouse GPC3 protein, S001-NB150-20(cyno+) and S001-NB152-101 do not bind to mouse GPC3 protein, and the other four VHH-hFc antibodies bind well to mouse GPC3 protein.

TABLE 16
Binding reactions of VHH-hFc to mouse GPC3 protein by ELISA

| | OD450 | | | | | | | |
|---|---|---|---|---|---|---|---|---|
| | Antibody concentration (nM) | | | | | | | |
| Antibody | 100 | 10 | 1 | 0.1 | 0.01 | 0.001 | 0.0001 | Blank |
| S001-NB150-20(cyno+) | 0.837 | 0.293 | 0.074 | 0.053 | 0.049 | 0.05 | 0.05 | 0.052 |
| S001-NB150-28(cyno+) | 2.32 | 2.32 | 2.098 | 1.704 | 0.554 | 0.114 | 0.067 | 0.054 |
| S001-NB150-56 | 2.605 | 2.238 | 1.964 | 1.265 | 0.29 | 0.08 | 0.053 | 0.05 |
| S001-NB152-1 | 0.324 | 0.55 | 0.461 | 0.153 | 0.059 | 0.049 | 0.048 | 0.048 |
| S001-NB152-101 | 0.288 | 0.073 | 0.115 | 0.19 | 0.23 | 0.222 | 0.109 | 0.046 |
| S001-NB152-72-2 | 2.479 | 2.336 | 2.262 | 1.54 | 0.278 | 0.063 | 0.042 | 0.039 |
| VH001 | 2.179 | 1.123 | 0.07 | 0.066 | 0.05 | 0.051 | 0.052 | 0.05 |
| Tab003 | 0.073 | 0.051 | 0.046 | 0.047 | 0.046 | 0.049 | 0.048 | 0.051 |
| Tab005 | 2.191 | 2.039 | 1.385 | 1.405 | 0.304 | 0.088 | 0.056 | 0.051 |
| hIgG1 | 0.075 | 0.05 | 0.048 | 0.05 | 0.047 | 0.05 | 0.052 | 0.051 |

(B) Binding of VHH-hFc to Cells Expressing Monkey GPC3 Protein by ELISA

HEK293T-monkey GPC3 cells were subjected to the FACS assay and data analysis according to the method described in Example 4, section (B). The analysis results are shown in Table 17 and FIGS. 12A-12B. The results show that all VHH-hFc bind to HEK293T-monkey-GPC3 cells, but do not bind to HEK293T cells.

TABLE 17
Binding reactions of VHH-hFc to HEK293T-monkey GPC3 cells and
HEK293T cells by FACS

| | HEK293T-monkey | | | |
|---|---|---|---|---|
| | GPC3 | | HEK293T | |
| | Maximum | | Maximum | |
| | mean | | mean | |
| | fluorescence | | fluorescence | |
| | intensity | EC50 | intensity | EC50 |
| Antibody | Max MFI | (nM) | Max MFI | (nM) |
| S001-NB150-20(cyno+) | 10355 | 57.56 | 961 | NB |
| S001-NB150-28(cyno+) | 12844 | 0.49 | 223 | NB |
| S001-NB150-56 | 1915 | 73.91 | 82 | NB |
| S001-NB152-1 | 15548 | 0.77 | 227 | NB |
| S001-NB152-101 | 19371 | 0.60 | 885 | NB |
| VH001 | 15551 | 0.23 | 404 | NB |
| S001-NB152-72-2 | 24678 | 0.58 | 136 | NB |
| Tab003 | 23225 | 2.85 | 484 | NB |
| Tab005 | 11875 | 0.19 | 581 | NB |
| hIgG1 | 107 | NB | 78 | NB |

### Example 6. Affinity for GPC3 Alpaca Antibodies

(A) Affinity of VHH-hFc Antibodies for Human GPC3 Protein

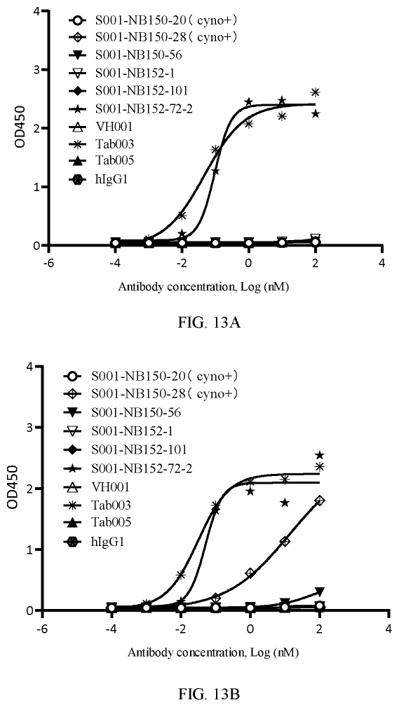

The anti-human GPC3 VHH-hFc was captured using a Protein A chip (GE Helthcare; 29-127-558). The sample and running buffer was HBS-EP+ (10 mM HEPES, 150 mM NaCl, 3 mM EDTA, 0.05% surfactant P20) (GE Healthcare; BR-1006-69). The flow cell was set at 25° C. The sample block was set at 16° C. Both were pretreated with the running buffer. In each cycle, first, the antibody to be tested was captured using the Protein A chip, and then antigen protein GPC3 at a single concentration was injected. The association and dissociation processes of the antibody with the antigen protein were recorded, and finally, the chip was regenerated using Glycine pH 1.5 (GE Healthcare; BR-1003-54). The association was determined by injecting different concentrations of human GPC3-His in the solution and maintaining for 240 s, wherein the flow rate was 30 μL/min, and the protein was serially diluted in a 1:1 dilution ratio from 200 nM (see detailed results for actual concentrations tested) to the 5th concentration. The dissociation phase was monitored for up to 600 s and triggered by switching from the sample solution to the running buffer. The surface was regenerated by washing with 10 mM glycine solution (pH 1.5) at a flow rate of 30 μL/min for 30 s. The difference in bulk refractive index was corrected by subtracting the responses obtained from the goat anti-human Fc surface. Blank injections were also subtracted (double reference). To calculate the apparent KD and other kinetic parameters, the Langmuir 1:1 model was used. The association rate (Ka), dissociation rate (Kdis), and binding affinity (KD) of VHH-hFc for human GPC3 protein are shown in the table, in which antibody Tab003 was used as a control. As shown in Table 18-1, Table 18-2, and FIGS. 13A-13B, the affinity of all VHH-hFc antibodies for human GPC3 protein was greater than 1E-7 M, the affinity of S001-NB150-28(cyno+), S001-NB152-101, S001-NB152-72-2, and VFIH-12 for human GPC3 protein was about 1E-8 M, the affinity of the other VHH-hFc antibodies for human GPC3 protein was greater than 1E-8 M, and the affinity of VH001 was about 1E-9 M.

TABLE 18-1
Binding affinity of VHH-hFc for human GPC3 protein

| Antibody | ka (1/Ms) | kd (1/s) | KD (M) |
|---|---|---|---|
| S001-NB150-20(cyno+) | 3.98E+05 | 2.41E−03 | 6.06E−09 |
| S001-NB150-28(cyno+) | 2.97E+05 | 5.74E−03 | 1.93E−08 |
| S001-NB150-56 | 1.04E+05 | 7.57E−04 | 7.25E−09 |
| S001-NB152-1 | 1.53E+05 | 1.14E−03 | 7.45E−09 |
| S001-NB152-101 | 1.51E+05 | 1.67E−03 | 1.11E−08 |
| S001-NB152-72-2 | 1.53E+05 | 1.78E−03 | 1.16E−08 |
| VH001 | 2.67E+05 | 4.43E−04 | 1.66E−09 |
| Tab003 | 2.62E+05 | 3.58E−04 | 1.36E−09 |

TABLE 18-2
Binding affinity of VHH-hFc for human GPC3 protein

| Antibody | ka (1/Ms) | kd (1/s) | KD (M) |
|---|---|---|---|
| VHH-12 | 1.44E+05 | 2.40E−03 | 1.67E−08 |
| Tab003 | 1.22E+05 | 4.26E−04 | 3.51E−09 |
| Tab005 | 2.08E+05 | 4.42E−05 | 2.13E−10 |

(B) Affinity of GPC3 Alpaca Antibodies for Monkey GPC3-his Protein

The affinity of VHH-hFc for monkey GPC3-His protein was determined according to the method described in Example 6, section (A), in which antibody Tab003 was used as a control. As shown in

TABLE 19
the affinity of VHH-hFc for monkey GPC3 protein was greater than
1E−8M.
Binding affinity of VHH-hFc for monkey GPC3 protein

| Antibody | ka (1/Ms) | kd (1/s) | KD (M) |
|---|---|---|---|
| S001-NB150-20(cyno+) | 2.43E+05 | 1.03E−03 | 4.23E−09 |
| S001-NB150-28(cyno+) | 3.06E+05 | 1.57E−03 | 5.12E−09 |
| S001-NB150-56 | 2.36E+05 | 1.09E−03 | 4.64E−09 |
| S001-NB152-1 | 1.75E+05 | 9.83E−04 | 5.63E−09 |
| S001-NB152-101 | 1.81E+05 | 4.80E−04 | 2.66E−09 |
| S001-NB152-72-2 | 2.34E+05 | 7.54E−04 | 3.23E−09 |
| VH001 | 3.52E+05 | 1.08E−03 | 3.06E−09 |
| Tab003 | 2.42E+05 | 3.09E−04 | 1.28E−09 |

(C) Affinity of GPC3 Alpaca Antibodies for Mouse GPC3-his Protein

The affinity of VHH-hFc for mouse GPC3-His protein was determined according to the method described in Example 6, section (A), in which antibody Tab005 was used as a control. As shown in Table 20, S001-NB150-20(cyno+) and S001-NB152-101 do not bind to mouse GPC3 protein, and the affinity of the other five VHH-hFc antibodies for mouse GPC3 protein was greater than 1E-7 M; among them, the binding affinity of VH001 for mouse GPC3 protein was about 1E-8 M, and the affinity of S001-NB150-28(cyno+), S001-NB150-56, S001-NB152-1, and S001-NB152-72-2 was greater than 1E-8 M.

TABLE 20
Binding affinity of VHH-hFc for mouse GPC3 protein

| Antibody | ka (1/Ms) | kd (1/s) | KD (M) |
|---|---|---|---|
| S001-NB150-20(cyno+) | No binding | | |
| S001-NB150-28(cyno+) | 1.71E+05 | 1.10E−03 | 6.42E−09 |
| S001-NB150-56 | 1.39E+06 | 1.29E−02 | 9.28E−09 |
| S001-NB152-1 | 2.03E+05 | 1.16E−03 | 5.74E−09 |
| S001-NB152-101 | No binding | | |
| S001-NB152-72-2 | 2.19E+05 | 8.72E−04 | 3.99E−09 |
| VH001 | 1.34E+05 | 1.37E−03 | 1.02E−08 |
| Tab005 | 5.24E+05 | 4.25E−05 | 8.13E−11 |

### Example 7. Analysis on Antigen-Binding Epitopes of Antibodies

(A) Identification of Antigen-Binding Regions of Antibodies

Mature GPC3 protein has a soluble amino-terminal (N-terminal) peptide of about 40 kD that can enter the blood and a membrane-bound C-terminal peptide of about 30 kD. Tab003 antibody recognizes a region at the C terminus of GPC3 protein that is near the cell membrane (membrane proximal end), while Tab005 antibody recognizes non-membrane-proximal regions. To identify whether the antigen-binding epitopes of VHH-hFc are located at the membrane proximal end, VHH-hFc was subjected to identification of membrane proximal binding by coating the polypeptide GC3pep (membrane proximal end) and the polypeptide YP7pep (membrane proximal end) of human GPC3 respectively according to the ELISA method described in Example 4, section (A). As shown in FIGS. 13A-13B and Table 21, antibody S001-NB152-72-2 binds to both polypeptide GC3pep and polypeptide YP7pep, and antibody S001-NB150-28(cyno+) binds to polypeptide YP7pep, both antibodies being antibodies that recognize membrane proximal epitopes; the other antibodies do not bind to polypeptide GC3pep and polypeptide YP7pep, and thus are non-membrane-proximal antibodies.

TABLE 21
Classification of membrane proximal and non-membrane-proximal
epitopes for VHH-hFc by ELISA

| | Binding region | |
|---|---|---|
| Antibody | GC3 pep | YP7pep |
| S001-NB150-20(cyno+) | − | − |
| S001-NB150-28(cyno+) | − | + |
| S001-NB150-56 | − | − |
| S001-NB152-1 | − | − |
| S001-NB152-101 | − | − |
| S001-NB152-72-2 | + | + |
| VH001 | − | − |

1. An antibody or an antigen-binding fragment specifically binding to GPC3, wherein the antibody or the antigen-binding fragment comprises a CDR1, a CDR2, and a CDR3 comprising an HCDR1, an HCDR2, and an HCDR3 selected from a VHH domain set forth in any one of SEQ ID NOs: 17-23 and 87.

2. The antibody or the antigen-binding fragment according to claim 1, wherein the HCDR1, the HCDR2, and the HCDR3 are determined according to the IMGT numbering system, the Kabat numbering system, or the Chothia numbering system, e.g., selected from Table 1; for example, the HCDR1 is selected from SEQ ID NOs: 24, 27, 30, 33, 36, 39, 42, 45, 48, 51, 54, 57, 60, 63, 66, 69, 72, 75, 78, 81, 84, 88, 91, and 94, the HCDR2 is selected from SEQ ID NOs: 25, 28, 31, 34, 37, 40, 43, 46, 49, 52, 55, 58, 61, 64, 67, 70, 73, 76, 79, 82, 85, 89, 92, and 95, and the HCDR3 is selected from SEQ ID NOs: 26, 29, 32, 35, 38, 41, 44, 47, 50, 53, 56, 59, 62, 65, 68, 71, 74, 77, 80, 83, 86, 90, 93, and 96;
preferably, according to the IMGT numbering system, the Kabat numbering system, or the Chothia numbering system, the HCDR1, the HCDR2, and the HCDR3 are selected from sequences set forth in SEQ ID NOs: 24-26, 27-29, and 30-32, respectively;
preferably, according to the IMGT numbering system, the Kabat numbering system, or the Chothia numbering system, the HCDR1, the HCDR2, and the HCDR3 are selected from sequences set forth in SEQ ID NOs: 33-35, 36-38, and 39-41, respectively;
preferably, according to the IMGT numbering system, the Kabat numbering system, or the Chothia numbering system, the HCDR1, the HCDR2, and the HCDR3 are selected from sequences set forth in SEQ ID NOs: 42-44, 45-47, and 48-50, respectively;
preferably, according to the IMGT numbering system, the Kabat numbering system, or the Chothia numbering system, the HCDR1, the HCDR2, and the HCDR3 are selected from sequences set forth in SEQ ID NOs: 51-53, 54-56, and 57-59, respectively;
preferably, according to the IMGT numbering system, the Kabat numbering system, or the Chothia numbering system, the HCDR1, the HCDR2, and the HCDR3 are selected from sequences set forth in SEQ ID NOs: 60-62, 63-65, and 66-68, respectively;
preferably, according to the IMGT numbering system, the Kabat numbering system, or the Chothia numbering system, the HCDR1, the HCDR2, and the HCDR3 are selected from sequences set forth in SEQ ID NOs: 69-71, 72-74, and 75-77, respectively;
preferably, according to the IMGT numbering system, the Kabat numbering system, or the Chothia numbering system, the HCDR1, the HCDR2, and the HCDR3 are selected from sequences set forth in SEQ ID NOs: 78-80, 81-83, and 84-86, respectively;
preferably, according to the IMGT numbering system, the Kabat numbering system, or the Chothia numbering system, the HCDR1, the HCDR2, and the HCDR3 are selected from sequences set forth in SEQ ID NOs: 88-90, 91-93, and 94-96, respectively.

3. The antibody or the antigen-binding fragment according to claim 1, wherein the CDR1, the CDR2, and/or the CDR3 comprises amino acid sequences having at most 10, 9, 8, 7, 6, 5, 4, 3, 2, or 1 mutation on the HCDR1, the HCDR2, and/or the HCDR3; the mutation is selected from an insertion, a deletion, and/or a substitution, and the substitution is preferably a substitution of conserved amino acids.

4. The antibody or the antigen-binding fragment according to claim 1, wherein the CDR1, the CDR2, and/or the CDR3 comprises sequences having at least 80%, 85%, 90%, 91%, 92%, 93%, 94%, 95%, 96%, 97%, 98%, 99%, or 100% identity to the HCDR1, the HCDR2, and/or the HCDR3, respectively.

5. The antibody or the antigen-binding fragment according to claim 1, wherein the antibody or the antigen-binding fragment comprises a single domain antibody comprising the CDR1, the CDR2, and the CDR3.

6. The antibody or the antigen-binding fragment according to claim 5, wherein the single domain antibody comprises a sequence set forth in any one of SEQ ID NOs: 17-23 and 87; optionally, the single domain antibody comprises a sequence having at least 80%, 85%, 90%, 91%, 92%, 93%, 94%, 95%, 96%, 97%, 98%, 99%, or 100% identity to the sequence set forth in any one of SEQ ID NOs: 17-23 and 87; or the single domain antibody comprises a sequence having at most 20, 19, 18, 17, 16, 15, 14, 13, 12, 11, 10, 9, 8, 7, 6, 5, 4, 3, 2, or 1 mutation compared with the sequence set forth in any one of SEQ ID NOs: 17-23 and 87; the mutation is selected from an insertion, a deletion, and/or a substitution, and the substitution is preferably a substitution of conserved amino acids.

7. The antibody or the antigen-binding fragment according to claim 1, wherein the single domain antibody comprises an FR region in a VHH domain set forth in any one of SEQ ID NOs: 17-23 and 87; optionally, the single domain antibody comprises a sequence having at least 80%, 85%, 90%, 91%, 92%, 93%, 94%, 95%, 96%, 97%, 98%, 99%, or 100% identity to the FR region in the VHH domain set forth in any one of SEQ ID NOs: 17-23 and 87; or the single domain antibody comprises a sequence having at most 15, 14, 13, 12, 11, 10, 9, 8, 7, 6, 5, 4, 3, 2, or 1 mutation compared with the FR region in the VHH domain set forth in any one of SEQ ID NOs: 17-23 and 87; the mutation is selected from an insertion, a deletion, and/or a substitution, and the substitution is preferably a substitution of conserved amino acids;
optionally, wherein the antibody or the antigen-binding fragment is: (1) a chimeric antibody or a fragment thereof, (2) a humanized antibody or a fragment thereof, or (3) a full human antibody or a fragment thereof;
optionally, wherein the antibody or the antigen-binding fragment comprises or does not comprise an antibody heavy chain constant region; optionally, the antibody heavy chain constant region may be selected from human, alpaca, mouse, rat, rabbit, and sheep; optionally, the antibody heavy chain constant region may be selected from IgG, IgM, IgA, IgE, and IgD, and the IgG is selected from IgG1, IgG2, IgG3, and IgG4; optionally, the heavy chain constant region is selected from an Fc region, a CH3 region, or a complete heavy chain constant region, preferably, the heavy chain constant region is a human Fc region, and more preferably has an amino acid sequence set forth in SEQ ID NO: 1; preferably, the antibody or the antigen-binding fragment is a heavy chain antibody;
optionally, wherein the antibody or the antigen-binding fragment is further conjugated to a therapeutic agent or a tracer; preferably, the therapeutic agent is selected from a radioisotope, a chemotherapeutic agent or an immunomodulator, and the tracer is selected from a radiocontrast medium, a paramagnetic ion, a metal, a fluorescent label, a chemiluminescent label, an ultrasound contrast agent, and a photosensitizer;
optionally, wherein the antibody or the antigen-binding fragment specifically binds to human, monkey, and/or murine GPC3; preferably, the antibody or the antigen-binding fragment binds to human, monkey, and/or murine GPC3 with a KD greater than 1.00E-7 M, 1.00E-8 M, 2.00E-8 M, 3.00E-8 M, 4.00E-8 M, 5.00E-8 M, 6.00E-8 M, 7.00E-8 M, 8.00E-8 M, 9.00E-8 M, 1.00E-9 M, 2.00E-9 M, 3.00E-9 M, 4.00E-9 M, 5.00E-9 M, 6.00E-9 M, 7.00E-9 M, 8.00E-9 M, 9.00E-9 M, or 1.00E-10 M.

8-11. (canceled)

12. A polypeptide, comprising the antibody or the antigen-binding fragment according to claim 1, wherein preferably, the polypeptide is further linked to an additional functional molecule, and preferably, the additional functional molecule is selected from one or more of: a signal peptide, a protein tag, an additional antigen-binding molecule, and a cytokine;
optionally, wherein the additional antigen-binding molecule specifically binds to an antigen other than GPC3 or binds to a GPC3 epitope different from that of the antibody or the antigen-binding fragment according to claim 1;
preferably, the antigen other than GPC3 is selected from: CD3, preferably CD3ε; CD16, preferably CD16A; NKG2D; CD40; 4-1BB; CD137 or CD19; EGFR; EGFRvIII; mesothelin; HER2; EphA2; Her3; EpCAM, MUC1; MUC16; CEA; Claudin18.2; a folate receptor; Claudin6; WT1; NY-ESO-1; MAGE3; and ASGPR1 or CDH16;
preferably, the additional antigen-binding molecule is an antibody or antigen-binding fragment;
preferably, the polypeptide is a multispecific antigen-binding molecule, and the multispecific antigen-binding molecule is bispecific, trispecific, or tetraspecific, and more preferably, the multispecific antigen-binding molecule is divalent, tetravalent, or hexavalent;
optionally, wherein the cytokine is selected from IL2, IL-6, IL-12, IL-15, IL-21, IFN, or TNF-alpha.

13-14. (canceled)

15. A chimeric antigen receptor (CAR), comprising an extracellular antigen-binding domain, a transmembrane domain, and an intracellular signaling domain, wherein the extracellular antigen-binding domain comprises the antibody or the antigen-binding fragment according to claim 1.

16. An immune effector cell expressing the chimeric antigen receptor according to claim 15, or comprising a nucleic acid fragment encoding the chimeric antigen receptor according to claim 15; wherein preferably, the immune effector cell is selected from a T cell, a natural killer cell (NK cell), a natural killer T cell (NKT cell), a double negative T cell (DNT cell), a monocyte, a macrophage, a dendritic cell, and a mast cell, and the T cell is preferably selected from a cytotoxic T cell, a regulatory T cell, and a helper T cell; and preferably, the immune effector cell is an autoimmune effector cell or an allogeneic immune effector cell.

17. An isolated nucleic acid fragment encoding the antibody or the antigen-binding fragment according to claim 1.

18. A vector, wherein the vector comprises the nucleic acid fragment according to claim 17.

19. A host cell, comprising the vector according to claim 18, wherein preferably, the cell is a prokaryotic cell or a eukaryotic cell, such as a bacteria (Escherichia coli), a fungus (yeast), an insect cell, or a mammalian cell (a CHO cell or a 293T cell).

20. A method for preparing the antibody or the antigen-binding fragment according to claim 1, comprising:
culturing a cell, and
isolating an antibody or an antigen-binding fragment expressed by the cell, or isolating a polypeptide expressed by the cell,
wherein the cell is a host cell comprising an isolated nucleic acid fragment, and the isolated nucleic acid fragment encodes the antibody or the antigen-binding fragment according to claim 1.

21. A method for preparing an immune effector cell, comprising introducing a nucleic acid fragment encoding the chimeric antigen receptor according to claim 15 into the immune effector cell, wherein optionally, the method further comprises initiating expression of the chimeric antigen receptor according to claim 15 in the immune effector cell.

22. A pharmaceutical composition comprising the antibody or the antigen-binding fragment according to claim 1, wherein optionally, the pharmaceutical composition further comprises a pharmaceutically acceptable carrier, diluent or adjuvant; and optionally, the pharmaceutical composition further comprises an additional antineoplastic agent.

23. A method for treating a GPC3-positive tumor or cancer, comprising: administering to a subject an effective amount of the antibody or the antigen-binding fragment according to claim 1, wherein preferably, the GPC3-positive tumor or cancer is selected from liver cancer, gastric cancer, lung cancer, breast cancer, head and neck cancer, bladder cancer, ovarian cancer, cervical cancer, kidney cancer, pancreatic cancer, cervical cancer, liposarcoma, melanoma, adrenal carcinoma, neurilemmoma, malignant fibrous histiocytoma, and esophageal cancer; more preferably, the GPC3-positive tumor or cancer is selected from liver cancer, gastric cancer, lung cancer, and breast cancer.

24-25. (canceled)

26. A kit, comprising the antibody or the antigen-binding fragment according to claim 1.

27. A method for detecting GPC3 expression in a biological sample, comprising contacting the biological sample with the antibody or the antigen-binding fragment according to claim 1 in a condition allowing formation of a complex between the antibody or the antigen-binding fragment and GPC3, wherein preferably, the method further comprises detecting the formation of the complex, and indicating the presence or an expression level of GPC3 in the sample.

28. A method for preparing a GPC3 assay reagent, wherein the method comprises using the antibody or the antigen-binding fragment according to claim 1.
